(19) United States
(12) Patent Application Publication
(10) Pub. No.: US 2025/0040960 A1
(43) Pub. Date: Feb. 6, 2025

(54) Percutaneous Blunt Dissection Access System and Method

(71) Applicant: ACCESSOR MEDICAL, LLC

(72) Inventors: Matthew Czar TAON, San Antonio, TX (US); Ian C. Barreto, Cerritos, CA (US); Raj Patel, Boise, ID (US); Jason S. Kim, Converse, TX (US); Tylor D. Connor, San Antonio, TX (US)

(21) Appl. No.: 18/229,154

(22) Filed: Aug. 1, 2023

(51) Int. Cl. A61B 17/34; A61B 17/32; A61B 90/00

(52) U.S. Cl. CPC A61B 17/3423; A61B 17/320016; A61B 17/3462; A61B 17/3496; A61B 90/39; A61B 2017/00557; A61B 2017/320044; A61B 2017/3486; A61B 2090/3966

(57) ABSTRACT

A percutaneous pathway system and method are disclosed with a sheath, a spacer upon the sheath, a needle parallel within the sheath, the needle is sheathed so the needle is not exposed beyond the spacer, a passthrough wherein the needle is retractable within the passthrough, the needle is not exposed beyond the spacer while retracted, the needle is extendable beyond the spacer to suitably acquire a target, the spacer provides a balloon, and an insufflation port on the sheath to insufflate the balloon of the spacer. A method for commencing an interventional radiology procedure provides inserting a needle, a balloon spacer, and a sheath percutaneously without initially extending the needle; expanding the balloon spacer while simultaneously maintaining the needle in sheathed position within the sheath; and subsequently unsheathing the needle through the sheath percutaneously.

### BACKGROUND OF THE INVENTION

### 1. Field of Invention

The present invention relates to the field of image guided, percutaneous surgery, including interventional radiology to access tissue in the abdomen and pelvis for biopsies, drain placement, and tumor ablation.

### 2. Description of the Related Art

Percutaneous devices can be used to perform numerous types of minimally invasive, image guided procedures including biopsies to sample tissues to diagnose lesions, drainage catheter placements to remove infectious or space-occupying fluid collections, or ablation needle placement to treat tumors. However, conventional percutaneous procedures typically require an access window in order to place a percutaneous device, meaning for example that if there is bowel surrounding an abscess or a solid organ obscuring access to the target, the procedure may not be able to be performed or worse is deemed contraindicated due to risk of puncturing an organ or damaging tissue.

Although some limited methods are occasionally used to displace tissue which is blocking access to an anatomic target (i.e., an abscess, a tumor, or a fluid collection), which include instillation of saline, air, use of an angioplasty balloon-such conventional methods are severely limited, because they are not controllable to the extent of surgical precision. Numerous problems can occur in such conventional situations: saline can flow to dependent portions of the body; or air can fill non-dependent portions of the body. Angioplasty balloons are not meant to accommodate biopsy needles, ablation needles, or drainage catheters.

In addition, conventional balloon dissectors are standalone. Such balloon dissectors are sometimes used to separate physical space from tissue. However, these conventional balloon dissectors are limited because such conventional balloons are not designed to facilitate other percutaneous interventional procedures such as drain placement, biopsies, or ablation needle placement. Specifically, when seeking to acquire a target for surgery, use of traditional balloon dissectors are not suited for nor designed to accommodate any coaxial techniques to permit minimally invasive, percutaneous biopsy needles, multipurpose drainage catheters, or ablation needles.

It is known that biopsy needles, drains, and ablation needles each have proximal hubs of varying sizes and shapes, as well as varying requirements for dwell time within the body. For example, biopsy needles can vary in length, for example, ranging from 10 cm to 20 cm in length. Such biopsy needles only remain in the body for a short period of time. In contrast, percutaneous drains have lengths of 25 cm and 40 cm and may remain indwelling within the abdomen or pelvis for days or weeks at time, thereby requiring removal of a peel-away access sheath and leaving the drain in place. Lastly, ablation needles have varying lengths of ablative distal tips. However, there is no system or method to provide a versatile sheath that can reduce or avoid damage by such biopsy needles, drains, or ablation needles.

Based on the limitations in the related art, it can be seen that there is a need to reduce and avoid tissue damage in conventional methods. More importantly, there is a critical need to overcome contraindications of conventional surgical risk in interventional radiology. This is particularly true where interventional surgeons would avoid performing surgery on a patient due to contraindications such as the belief that puncturing an organ is too risky. In this regard, more deeply, it can also be seen that there is a need for a system and method for a safe system and method to reach an otherwise unreachable organ directly.

Further, based on the limitations in the related art, it can be seen that a percutaneous blunt dissection of tissue is needed to firstly create pathways to anatomic targets, which would not be previously accessible due to overlying organs or anatomic tortuosity, and then to, safely near the target site access and create a pathway to deploy the needed surgical tool from the point of safety after creating a safe space. It can be seen that systems and methods to create surgical deployment from an inner, percutaneous safe space within the body has not been addressed by any traditional surgical methods. This is especially true where pre-surgical imaging shows tissues or organs that block or obscure a target area for surgery, such as an abscess, lesion, tumor, fluid, or any other undesirable deposit or substance in the body. It can be seen that despite conventional standards in medicine, believing that damage to tissue or organs upon entry into the body cavity will necessarily occur and therefore bar surgery is in fact wrong, and with further investigation ethical medicine demands pursuit of healing and treatment where patients consent and interventional radiologists are capable using a system and method enabling safer reach beyond a blocking organ by spacing and reaching the target area without damaging tissue or other organs otherwise blocking access to the target.

In addition, based on the limitations in the related art, it can also be seen that there is a need for sheath placement in regards to pathway creation, in particular, to create a passageway for subsequent deployment therefrom with the appropriate needed tool. It can be seen that such tools include biopsy needles, drainage catheters, or ablation needles to reach anatomic targets within the abdomen and pelvis. Finally, it can be seen that there is a need for a system and method for percutaneous blunt dissection of tissue to create pathways to anatomic targets, which would not be previously accessible due to overlying organs or anatomic tortuosity.

### SUMMARY OF THE PRESENT INVENTION

The present invention seeks to resolve long-held beliefs that many surgical operations are contraindicated. The present invention seeks to resolve the shortcomings of conventional surgical systems and methods as explained in the prior background section. This will be accomplished by the change and addition of certain features.

A percutaneous pathway system is disclosed. The system can have a peel-away coaxial sheath and percutaneous needle access device, with separately inflatable balloons on each half of the peel-away sheath, suitable for obtaining percutaneous access into the abdomen and pelvis with subsequent blunt tissue dissection and/or tissue manipulation.

An aspect of the present invention is to use a combination of techniques in a system that employs a peel-away tool, a balloon-mediated, blunt dissection approach, a sheath for both peel-away adhesion as well as simultaneous needle access without causing undue tissue damage.

Another aspect of the present invention is to provide an effective system and method for image-guided, percutaneous, surgery, including interventional radiology, which requires accessing tissue in the abdomen and pelvis for minimally-invasive, biopsies, drain placement, and tumor ablation. Related aspects include radiographic and echogenic properties to coincide with a multi-step, organ-safety-focused approach.

It is an object of the invention to provide a system that allows percutaneous, coaxial placement of minimally invasive surgical devices, such as biopsy needles, drainage catheters, or ablation needles, and to blunt dissect any overlying tissue or obscuring abdominopelvic organs by the use of a balloon dissection sheath.

It is a further object of the invention to provide a peel-away, coaxial platform by which the balloon dissection sheath can be removed over a multipurpose drainage catheter or ablation needle, without disrupting the position of the needle or losing target access.

It is another object of the invention to provide a steerable, coaxial platform by which safe, blunt dissection and directionality can be performed for precise placement of minimally invasive, percutaneous devices.

A first aspect of the present invention is to provide a medical device with a peel-away sheath can provide a protected, guided access pathway for placement and removal of needles and probes. The pathway can be tubular, coaxial but are not necessarily so; the present invention is not limited strictly to coaxial or tubular embodiments as other pathways can be made without a full tube or a strictly coaxially concentric tube as other shapes providing a pathway can easily foreseeably be formed and also apply within the scope and teachings of the present invention. Once the needles and/or probes have reached their target tissue for biopsy, drain placement, or tissue ablation, the sheath can be peeled away so that it accommodate a variety of biopsy needle, drain, and ablation needle lengths.

A second, separate aspect of the present invention is to provide a medical device having two balloon access insufflation ports which inflate to a predetermined size and shape for dissection tissue layers for the purpose of conducting a desired percutaneous procedure. It is unique to have two, separate insufflation ports and two separate balloons on a single sheath since the balloons can function both for tissue dissection and still be removed via the previously described peel-away methodology.

A third, separate aspect of the present invention is to provide a medical device which facilitates single access percutaneous blunt dissection, followed by immediate drainage catheter placement, without the requirement of wire placement to preserve access or exchange of devices. Percutaneous blunt dissection provides atraumatic access to a desired target tissue. However, the desired surgical tools must still be able to be advanced through the blunt dissection device. One preferred configuration for percutaneous drainage catheter placement is to have a percutaneous drainage catheter, metallic trocar, and sharp needle stylet positioned within the peel-away, dual balloon sheath, so that once blunt dissection has positioned the sheath tip immediately at the target site, the drainage catheter and needle can be immediately advanced into the target abscess.

A fourth, separate aspect of the present invention is a peel away sheath with a Luer lock tip and hemostatic valve, this allows placement of any device without leakage of intraabdominal or intrapelvic contents or introduction of air from the environment into the abdomen/pelvis.

A fifth, separate aspect of the present invention is the relatively stiff sheath with more malleable distal tip portion to allow for advancement of the sheath, without kinking, while also allowing bending of the distal tip in case steerable needles are required.

A sixth, separate aspect of the present invention is the capability to use steerable needles to manipulate the balloon-mediated blunt dissecting tip to facilitate access to an anatomic target.

A seventh, separate aspect of the present invention is the ability to utilize different modalities to blunt dissect tissue once percutaneous access has been obtained. Specifically, a blunt-dissecting tip may be comprised of metallic components such as nitinol, a woven mesh material, and/or plastic such as polyurethane, to facilitate blunt dissection for the purpose of moving overlying bowels or organs and facilitate a desired surgical procedure.

An eighth, separate aspect of the present invention is two medical balloon devices, with separate insufflation ports, which unfold, evert, and/or inflates in a controlled, predetermined manner. Various shapes and sizes can be developed and utilized to facilitate a desired surgical procedure.

A ninth, separate aspect of the present invention are blunt dissection methods for dissecting tissue to provide access to an anatomical structure inside the patient's abdomen and pelvis.

A tenth, separate aspect of the present invention is a configuration using a stiff metallic shaft, with a coaxial balloon, atraumatic nitinol mesh, woven mesh material, and/or atraumatic plastic tip, to facilitate blunt dissection through a conventional peel-away sheaths. The stiff metallic shaft allows coaxial traversal through existing peel-away sheaths, while the balloon, atraumatic nitinol mesh, woven mesh, or atraumatic plastic tip facilitates blunt dissection. Once the blunt dissection tip is at the target tissue, along with the conventional peel-away sheath, the blunt dissection tip can be removed and exchanged for a different device to facilitate a desired surgical procedure such as biopsy, drain placement, or ablation.

An eleventh, separate aspect of the present invention is the integration of sonographic, fluoroscopic, and CT markers at the tip and along the shaft of the device to facilitate image-guided percutaneous placement and usage of this device. This is critical to ensure maximum effectiveness in the device's use for image guided percutaneous surgeries.

### BRIEF DESCRIPTION OF THE DRAWINGS

Non-limiting exemplary embodiments are provided in the drawings as follows:

FIG. 1 (Prior Art) shows a blunt coaxial stylet.

FIG. 2 (Prior Art) shows a needle;

FIG. 3 (Prior Art) shows a trocar;

FIG. 4 (Prior Art) shows a drainage catheter;

FIG. 5 (Prior Art) shows a peel-away sheath without balloon insufflation;

FIG. 6 (Prior Art) shows a peel-away sheath without a side port;

FIG. 7 (Prior Art) shows a percutaneous biopsy needle;

FIG. 8 (Prior Art) shows a percutaneous needle ablation probe;

FIG. 9 (Prior Art) shows a robotic percutaneous needle insertion device, exemplified by U.S. Pat. Nos. 8,663,130; 11,039,891; 11,202,684; and CN105813585;

FIG. 10 (Prior Art) shows a robotic percutaneous needle insertion device, typified by WIPO Pub. No. WO2022195210A1 filed Mar. 14, 2022;

FIG. 11 shows an exploded view of a possible embodiment of the present invention with peel-away, balloon tipped sheath with inner pigtail drainage catheter, pre-loaded onto a metallic trocar and sharp, inner access needle;

FIG. 12 shows a view of a possible embodiment of the present invention the coaxial peel-away, balloon tipped sheath with inner pigtail drainage catheter, pre-loaded onto a metallic trocar and sharp, inner access needle;

FIG. 13 shows a secondary configuration view of a possible embodiment of the present invention with balloon-tipped, peel-away sheath; and insufflation ports located along the shaft of the peel-away sheath rather than along the hubs;

FIG. 14 shows a non-insufflated, sheathed configuration of a possible embodiment of the present invention;

FIG. 15 shows a sheathed configuration with insufflated balloons of an insufflated configuration of a possible embodiment of the present invention;

FIG. 16 shows a cut-away view of the balloon-tipped, peel away sheath with coaxial internal drainage catheter, metallic stiffener trocar, sharp inner needle, and insufflation tubing of an embodiment of the present invention;

FIG. 17 shows a multilevel cutaway with view of trocar with inner needle exposing its pointed tip therein of an embodiment of the present invention;

FIG. 18 shows a multilevel cut-away view of the shaft of the device displaying the coaxial nature of an embodiment of the present invention;

FIG. 19 shows a cross sectional view of the device shaft depicting the coaxial components of an embodiment of the present invention;

FIG. 20 shows a cut away view along the shaft of the pigtail drainage catheter depicting the coaxial nature of an embodiment of the present invention;

FIG. 21 shows an exploded view of the peel-away, balloon tipped sheath, with the inner drainage pigtail catheter positioned alongside the coaxial metallic inner stiffener, and sharp inner metallic needle;

FIG. 22 shows compatibility of the peel-away balloon tipped sheath with a percutaneous biopsy needle device (FIG. 7);

FIG. 23 shows compatibility of the peel-away balloon tipped sheath with an ablation needle probe (FIG. 8);

FIG. 24 shows a cross sectional view of an embodiment of the present invention with peel away sheath insufflated balloons as spacers used coaxially with a pigtail catheter, metallic inner stiffener trocar, and sharp metallic needle;

FIG. 25 shows a cross sectional view of the peel away sheath with insufflated balloons used coaxially with a pigtail catheter, metallic inner stiffener trocar, and sharp metallic needle;

FIG. 26 shows various shapes of a balloon-based version of spacer in a possible embodiment of the present invention;

FIG. 27 shows various shapes of a balloon-based version of spacer in a possible embodiment of the present invention;

FIG. 28 shows various shapes of a balloon-based version of spacer in a possible embodiment of the present invention;

FIG. 29 is a view of a possible embodiment of the present invention with different shaped balloon tip with the coaxial drain, hollow metallic stiffener trocar, and sharp metallic needle;

FIG. 30 shows four examples of stages of alternative possible embodiments of the present invention configured for blunt dissection using a shaft and blunt dissection tip;

FIG. 31A shows a possible embodiment of the present invention with steerability by which the curve of access sheath can be manipulated to safely access any anatomic target. As shown in FIG. 31A, there can be an absence of balloon-dissecting tip spacer;

FIG. 31B shows a possible embodiment of the present invention with steerability with presence of balloon-dissecting tip spacer by which the curve of access sheath 1101 can be manipulated to safely access any anatomic target;

FIG. 32A shows a possible embodiment of the present invention with insufflation port and the absence of a peel away sheath;

FIG. 32B shows a possible embodiment of the present invention with insufflation port with peel away sheath;

FIG. 33 shows a possible embodiment of the present invention configured with an unsheathed, sharp needle to facilitate access into the abdominopelvic cavity, coaxial hollow metallic stiffener trocar, pigtail drainage catheter, peel-away, blunt dissection sheath, with no balloon insufflation;

FIG. 34 shows a possible embodiment of the present invention configured with a preferred embodiment configuration for blunt dissection, with the sharp needle tip withdrawn into the hollow metallic stiffening trocar and peel-away, balloon-tipped sheath;

FIG. 35 shows a possible embodiment of the present invention where, after overlying tissue has been blunt dissected away from the anatomic target, this configuration shows coaxial advancement of the sharp needle tip, metallic stiffener trocar, and pigtail drainage catheter;

FIG. 36 shows a possible embodiment of the present invention configured with metallic stiffener trocar and sharp needle tip are slightly withdrawn and the pigtail, multi-side hole, drainage catheter is shaped within the target fluid collection;

FIG. 37 shows a possible embodiment of the present invention configured with the sharp needle, hollow stiffening trocar, and peel-away blunt dissection sheath have been removed, leaving only the drainage catheter within the target fluid collection;

FIG. 37 shows an image-guided view, pre-procedure, of an abscess with cecum and small bowel;

FIG. 38 is a diagram of an image-guided view of pre-procedure conditions with callouts marked for an abscess (I), cecum (II) and small bowel (III);

FIG. 39 shows a diagram of an image-guided view of pre-procedure conditions;

FIG. 40 shows a diagram of an image-guided view of an embodiment of the present invention;

FIG. 41 shows a diagram of an image-guided view of an embodiment of the present invention;

FIG. 42 shows a diagram of an image-guided view of a sharp tipped stylet with multipurpose drain of an embodiment of the present invention at an acquired target;

FIG. 43 shows a diagram of an image-guided view with multipurpose drain catheter;

FIG. 1000 demonstrates an example utilization of the device for an intra-abdominal abscess surrounded by multiple loops of bowel, and the first procedural step using the peel-away balloon tipped catheter in a possible embodiment of the present invention;

FIG. 1001 demonstrates inflation of the balloon-dissecting tip, enabling blunt dissection and safe advancement of the device towards the abscess in a possible embodiment of the present invention;

FIG. 1002 shows advancement of a sharp coaxial needle once blunt dissection has moved nontarget tissue out of the way and the tip of the sheath can be immediately adjacent to the target tissue in a possible embodiment of the present invention;

FIG. 1003 shows advancement of the pigtail catheter into the target tissue or target abscess in a possible embodiment of the present invention;

FIG. 1004 shows deflation of spacers can facilitate the peel-away removal of the sheath in a possible embodiment of the present invention;

FIG. 1005 shows a further step of peel away removal of sheath in a possible embodiment of the present invention;

FIG. 1006 shows a subsequent step with drainage catheter present a possible embodiment of the present invention;

FIG. 1007 shows ultrasound guided access of target tissue using peel-away, balloon tip sheath in a possible embodiment of the present invention;

FIG. 1008 demonstrates compatibility of the blunt dissection sheath with robotic or artificial-intelligence devices (e.g., FIG. 9) in a non-limiting anatomical example demonstrating a possible embodiment of the present invention; and

FIG. 1009 demonstrates compatibility of the blunt dissection sheath with an additional robotic or artificial-intelligence device (e.g., FIG. 10) in a non-limiting anatomical example demonstrating a possible embodiment of the present invention.

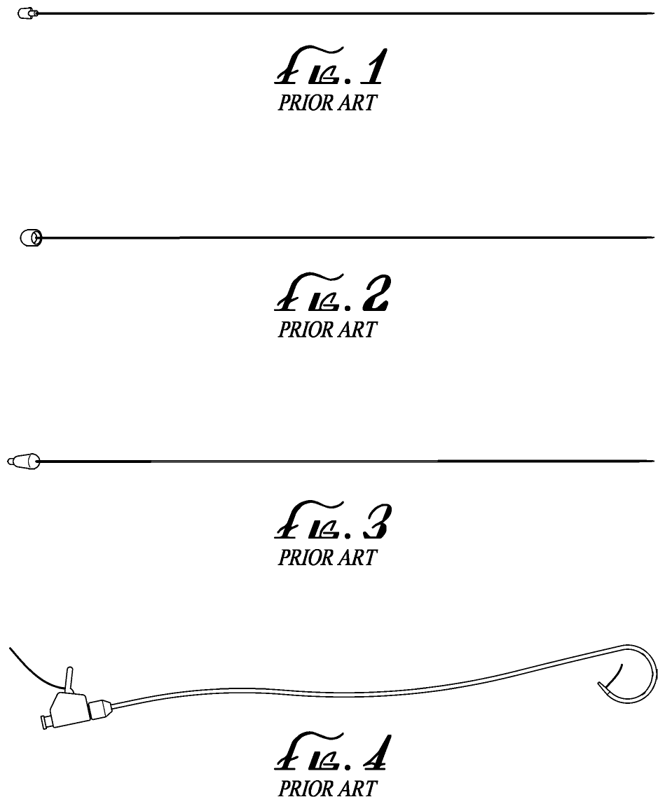

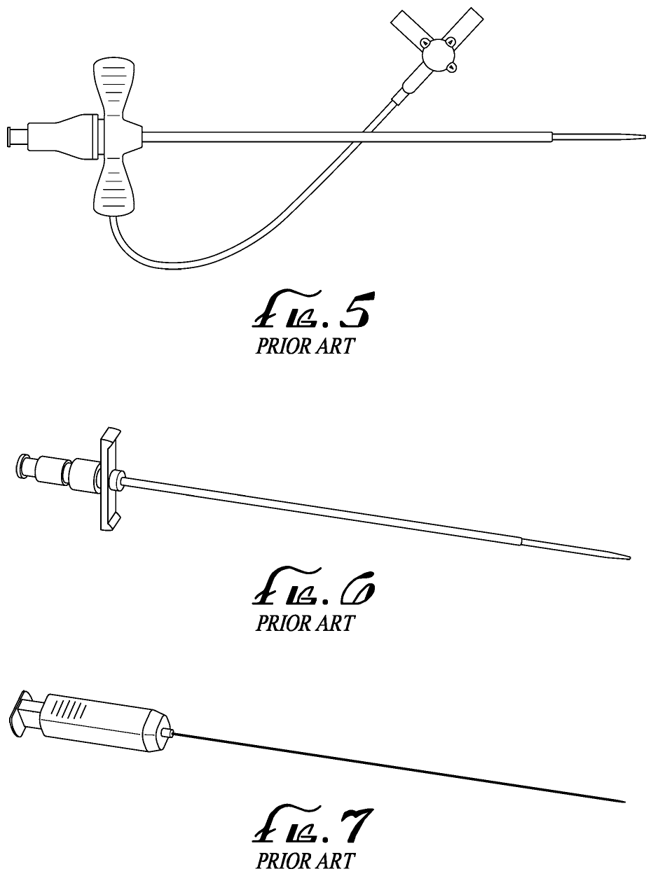

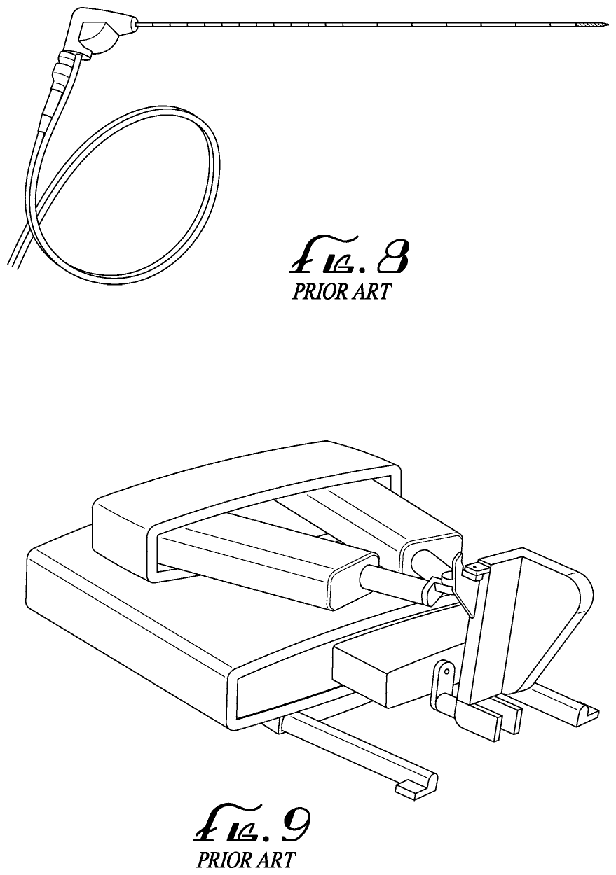

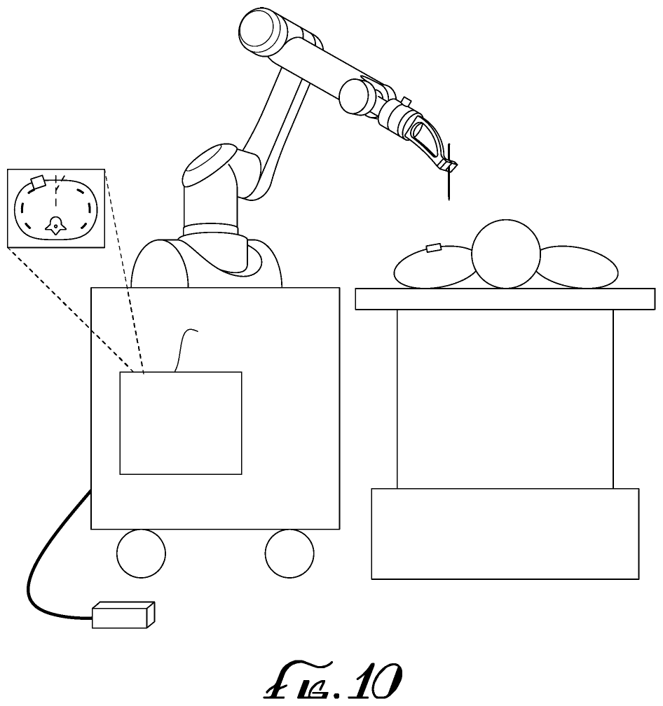

### DETAILED DESCRIPTION OF THE PREFERRED EMBODIMENTS

In the following description of the preferred embodiments, reference is made to the accompanying drawings that form a part hereof, in which is shown by way of illustration specific embodiments in which the invention may be practiced. It is to be understood that other embodiments may be utilized and structural changes may be made without departing from the scope of the present invention.

References throughout the specification to “interesting embodiment”; “possible embodiment”; “preferred embodiment”; “some embodiments”; “an embodiment”; and like reference to “embodiment” are non-limiting examples to aid in understanding the present invention. An “embodiment” provides that there can be one or more embodiments that can involve the given element or aspect of the invention. Thus, multiple instances of “an embodiment” and like reference do not necessarily refer to the same embodiment.

This specification provides for specific meanings with respect to the present invention, the meanings of which shall be understood as stated below. The following terms are provided and defined as follows:

“Abscess” refers to a localized area typically with swelling of body tissue, filled with purulent material, commonly known as pus.

“Afebrile” refers to a patient's state as being without a fever.

“Anesthesia” refers to a state of temporary induced loss of sensation or awareness, often induced using specific gases or drugs to ensure patient comfort during surgical procedures.

“Appendicitis” refers to inflammation and pus-filled state of the appendix, typically with abdominal pain.

“Appendicolith” refers to a hard, calcified deposit located within the appendix.

“Armamentarium” refers to a wide range of equipment, medicines, and techniques available to a healthcare practitioner for use in diagnosis and treatment.

“Broad-spectrum antibiotics” refer to antimicrobial antibiotics targeting multiple groups of pathogenic bacteria.

“Cannula” refers to a thin tube inserted into a vein or body cavity for administering medication, draining excess fluid, or facilitating the insertion of a surgical instrument.

“Catheter” refers to a flexible tube designed for insertion to enable drainage.

“Cecum” refers to a pouch-like structure located at the junction where the small intestine meets the large intestine.

“CT Scan” or Computerized Tomography Scan, is an imaging procedure used for diagnosing conditions like tumors, investigating internal bleeding, assessing internal injuries or damage, and for obtaining tissue or fluid samples for biopsy.

“CT gantry” refers to the cylindrical or ring-shaped structure of a CT scanner where the patient is positioned for a CT scan.

“Cryoablation” refers to a therapeutic process where extremely cold substances or instruments, such as a cryoprobe, are employed to freeze and subsequently destroy abnormal tissues.

“Hydrodissection” is a technique involving the injection of fluid under ultrasound guidance, primarily aimed at releasing tight fascia compressing a nerve.

“Hydropneumodissection” is a technique utilized to facilitate cryoablation of recurrent sarcoma adjacent to the sciatic nerve, primarily in a limb-sparing context.

“Iliopsoas muscle” is a major muscle group in the body, functioning primarily as a powerful hip flexor and assisting in the external rotation of the femur.

“Laparoscopic appendectomy” is a surgical procedure involving the removal of the inflamed appendix through small incisions, facilitated by the insertion of a long thin tube known as a laparoscope.

“Leukocytosis” is characterized by an elevated count of white blood cells in the bloodstream, often indicating an ongoing inflammatory response or infection.

“MRI” or Magnetic Resonance Imaging is a non-invasive imaging technique that generates detailed internal images of specific parts of the body, assisting physicians in diagnosis and evaluation of medical conditions.

“Percutaneous drainage” is described as a minimally invasive procedure used to extract fluid collections such as abscesses, commonly applied in regions like the abdomen and pelvis.

“Paracolic gutter” is defined as the anatomical space located between the colon and the lateral abdominal wall.

“Sarcoma” represents a type of malignancy that originates in bone and soft tissue structures.

“Sciatic nerve” refers to the major nerve that runs down the back of each leg.

The drawing figures provided herewith are non-limiting examples, not to be narrowly construed in light of the entire specification which can provide for significant variations consistent with the teaching herein to obtain benefits, including as claimed.

Turning to FIG. 11, this figure provides an exploded view of peel-away, balloon tipped sheath with inner pigtail drainage catheter, pre-loaded onto a metallic trocar and sharp, inner access needle.

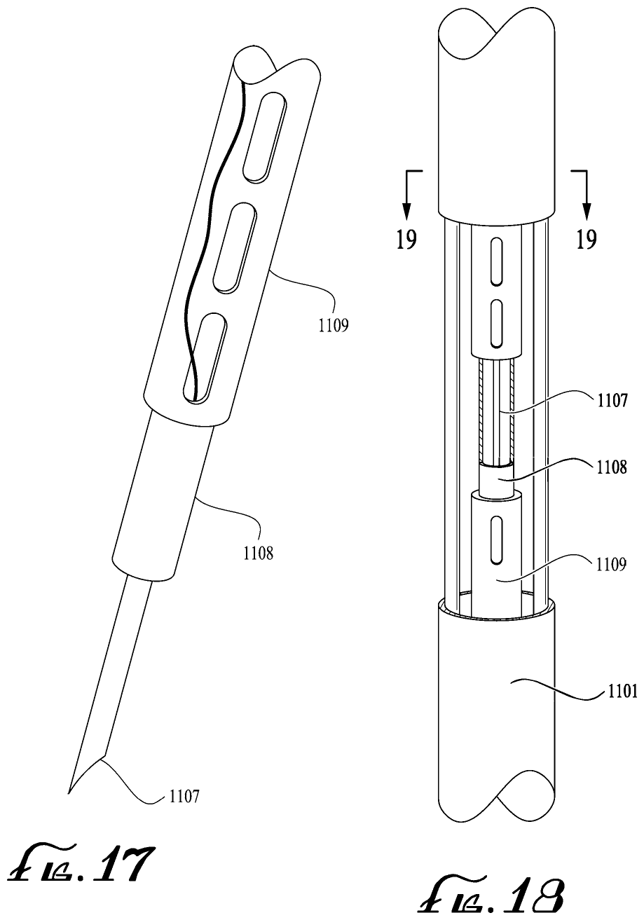

Shown in FIGS. 11 and 12 are sheath 1101, spacer 1102, insufflation port 1103, valve 1104, passthrough 1105, contrasting tip 1106, needle 1107, trocar 1108, catheter 1109, and retention suture 1110.

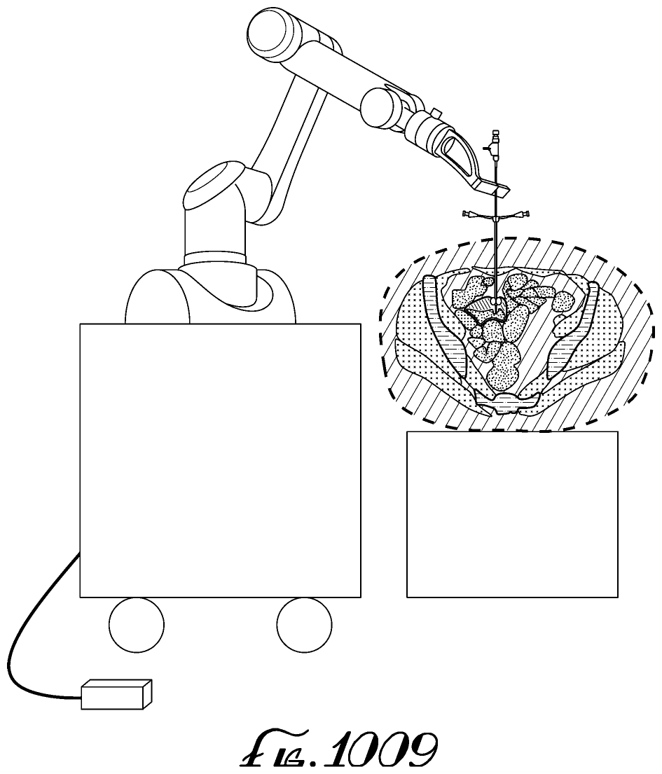

Sheath 1101 can be a peel-away sheath. Sheath 1101 can be a non-peel away sheath. Sheath 1101 can be a plastic sheath. Sheath 1101 can be composed of polyethylene, polypropylene, polyurethane, polycarbonate, polyethermide, pebax, or nylons. Sheath 1101 can be a metallic sheath. Sheath 1101 can be a hydrophilic or nonhydrophilic coating. Sheath 1101 size can range between 2 French to 16 French. Sheath 1101 lengths can range from 8 cm to 15 cm to accommodate a wide variety of biopsy needles, access needles, or percutaneous drains. Sheath 1101 can be advanced by a physician, machine-assisted with human intervention, or by a compatible robotic device (FIG. 1008). In some embodiments, portholes can be provided thereon for sheath flushing, particularly in cases where catheter 1109 can be used. In relation to sheath 1101, needle 1107 can be sheathed or unsheathed as exposed for use. However, use of spacer 1102 consistent with the present invention can safely provide protection for tissues or organs prior to deploying needle 1107 through sheath 1101 past spacer 1102. A benefit of sheath 1101 can be to facilitate removal of sheath 1101 once acquiring access to target. Sheath 1101 can be removably peeled. Sheath 1101 can be coaxial around passthrough 1105 where passthrough 1105 is an access tube.

Spacer 1102 can be atraumatic. Spacer 1102 can be an at least one balloon-spacer tip for blunt dissection. Spacer 1102 can be an atraumatic balloon. Spacer 1102 can be atraumatic. Spacer 1102 can be a noncompliant, semi-compliant, or compliant balloon. Spacer 1102 can be blunt. Spacer 1102 can be blunt dissection tip. Spacer 1102 can be a single balloon. Spacer 1102 can be, in some embodiments, composed of two or more tips. Spacer 1102 can be, in some embodiments, a metallic mesh. Spacer 1102 can be, in some embodiments, a plastic mesh. Spacer 1102 can embody different shapes. Spacer 1102 can be formed by an at least one insufflatable balloon. There can be a second spacer 1102 formed by a second insufflatable balloon. Spacer 1102 can be at a distal end of sheath 1101. In sheathed and unsheathed configurations, a benefit of the present invention with sheath 1101 can be to protect organs and tissue. Spacer 1102 can comprise an at least one dilute contrast or can be formed of contrasting material, whether in balloon, mesh or other variations. Spacer 1102 can have a contrast marker thereon. Spacer 1102 can be expandable. Spacer 1102 can be placed upon sheath 1101, preferably at a distal end of sheath 1101 near passthrough 1105 where needle 1107 can be retracted or extended therethrough.

Port 1103 herein generally refers to insufflation ports. Port 1103 can facilitate insufflation of spacer 1102, in balloon-based embodiments of the present invention. In some preferred embodiments, two separate insufflation ports can separately fill spacer 1102 as a balloon on each side to facilitate peel away capabilities of sheath 1101. A single balloon spacer 1102 and single insufflation port 1103 are possible configurations, which are useful in certain clinical scenarios. Port 1103 can be configured such that the present invention provides two or more separate insufflation ports 1103. Port 1103 in some embodiments can be comprised of a single insufflation port which fills one or more balloons. Port 1103 can be positioned along the shaft or hub of sheath 1101. Port 1103 can be composed of varying lengths. Port 1103 can have a three-way stop. Port 1103 can have a valve or lock to prevent air loss. Port 1103 can be compatible with fluid or air insufflation. In an embodiments, there can be an at least one or first insufflation port 1103 on sheath 1101; and in some embodiments, a second insufflation port 1103 on sheath in opposition to the first insufflation port 1103.

Valve 1104 can be suitably configured for the application of the surgical dissection tool deployed other than spacer 1102. In some preferred embodiments, valve 1104 can be a hemostatic valve, understood to be in the style of a hemostatic valve which operates in similar manner, but is not limited to blood-based applications. Valve 1104 can have a Luer Lock to allow flushing of sheath 1101. Valve 1104 can have an absence of a Luer Lock. Hemostatic valve 1104 can be comprised of a Luer Lock. Hemostatic valve 1104 can be comprised of a slip lock. Hemostatic valve 1104 can accommodate a rotating hemostatic valve. Valve 1104 can be on sheath 1101. A benefit of valve 1104 can be to facilitate flushing of sheath 1101.

Passthrough 1105 can be a portal, tube, diameter formed by a tube or other like shaped guided passageway. Passthrough 1105 can be, in some preferred embodiments, a coaxial capable inner diameter to suitable to accommodate access needles, biopsy devices, drainage catheters, or ablation needles. By way of non-limiting illustration, passthrough 1105 in some preferred embodiments can provide a coaxial inner diameter, for example within sheath 1101, and can accommodate 0.035 inch wires or 0.018 inch wires. In an embodiment, passthrough 1105 can have needle 1107. Needle 1107 can be retractable therein meaning within passthrough 1105. Passthrough 1105 can serve as an access tube within sheath 1101.

Contrasting tip 1106 can be any image-guided contrasting tip. In many preferred embodiments, contrasting tip can be a radiopaque tip or an echogenic tip to facilitate fluoroscopic, cone-beam CT, CT, or ultrasound-guided procedures. Metallic or otherwise dense material showing in radiographic or echogenic guided procedures are required. One having ordinary skill in the pertinent art would know how to implement the invention only after reading carefully and fully the disclosures and teachings of the present invention in full. Contrasting tip 1106 can be formed on needle 1107, which can be suitably configured for image-guided surgical procedures.

Needle 1107 can be a pointed tip needle. Needle 1107 can be echogenic or radiopaque tip to facilitate fluoroscopic, cone-beam CT, CT, or ultrasound-guided procedures. Needle 1107 can have various shapes to facilitate access into the abdominopelvic cavity. In some preferred embodiments, needle 1107 be formed as a sharp needle tip. Needle 1107 can be formed as a sharp, metallic needle. Needle 1107 be positioned inside trocar 1108. Needle 1107 can be rigid or flexible. In a preferred embodiment, needle 1107 can be a sharp inner access needle. In a preferred embodiment, needle 1107 can be positioned within and along hollow metallic stiffener trocar 1108. A benefit of needle 1107 within trocar 1108 can be to retract or expose the tip of needle 1107. Needle 1107 can be within sheath 1101. Needle 1107 can be sheathed so needle 1107 will not be exposed beyond spacer 1102 while retracted. Needle 1107 can be extendable beyond spacer 1102 to suitably acquire target. Needle 1107 can be parallel within sheath 1101.

Trocar 1108 can preferably be a metallic stiffener. Trocar 1108 can be hollow. Trocar 1108 can be coaxial within catheter 1109. Trocar 1108 can be rigid. Trocar 1108 can be flexible and shapeable. Trocar 1108 can be manipulated by turning, as torque-able or steerable. Trocar 1108 or a portion thereof can be positioned inside a portion of catheter 1109 consistent with the Figures. Catheter 1109, trocar 1108 and needle 1107 can be parallel or longitudinally parallel with sheath 1101, or within sheath 1101 or in any combination.

Catheter 1109 can be an insertable hollow tube. In many preferred embodiments, catheter 1109 can be a drainage catheter. In some preferred embodiments, catheter 1109 can be a pigtail, multi-sidehole, coaxial drainage catheter. In some embodiments, sideholes in catheter 1109 can facilitate drainage by providing vents along catheter 1109 (FIGS. 17 & 18). Catheter 1109 can be a multipurpose, looped, multi-sidehole, pigtail drainage catheter. Catheter 1109 can be pre-shaped to form a loop, without a pull-string used to shape catheter 1109. Catheter 1109 can also be shaped using a pull-string. In embodiments with catheter 1109 as multipurpose, looped, multi-side hole, pigtail drainage catheter 1109 can have varying diameters of the looped drainage tip. In some possible embodiments, catheter 1109 can be made of biocompatible material including but not limited to plastic, or more specifically, polytetrafluoroethylene (PTFE). A benefit of catheter 1109 as used with the present invention can be to provide a broad range of functionality, including drainage for an abscess or fluid where there can be an obstructing organ or tissue that would make it otherwise unreachable to conduct drainage. Catheter 1109 can be placed along or longitudinally parallel with sheath 1101.

Retention suture 1110 can be a pull-string connected to one end of catheter 1109. A benefit of retention suture 1110 can be capable of shaping catheter 1109 for example to deform or curl catheter 1109 to acquire a target. Retention suture (1110) can be within catheter (1109) and capable of curling catheter 1109. Retention suture 1110 can facilitate formation of a pigtail loop or additional shape of catheter 1109. Retention suture 1110 can be placed within catheter 1109, particularly in embodiments with catheter 1109 as a percutaneous drainage catheter. A benefit of retention suture 1110 can be to facilitate formation of a pigtail loop to assist with drainage via the catheter's side holes. Retention suture 1110 can run within the length of percutaneous drainage catheter and be capable of curling catheter 1109, for example, into a pigtail loop to assist with drainage via an at least one or more side hole of catheter 1109. There can be side holes on catheter 1109 (FIGS. 17 & 18).

FIG. 12 shows the coaxial peel-away, balloon tipped sheath with inner pigtail drainage catheter, pre-loaded onto a metallic trocar and sharp, inner access needle 1107. The balloons are insufflated. There are two balloon access ports to insufflate and fill the balloon, which functions to facilitate atraumatic percutaneous blunt dissection. There can be a separation delineation along the shaft of the sheath, which provides the critical function of allowing peel-away removal of the sheath upon acquiring target access. There can be a hemostatic valve at the hub to prevent blood or fluid backflow, but also serve as a Luer-lock flush for the sheath. There are two balloon insufflation hubs, which allows each balloon to be insufflated individually and also allows the sheath to have the peel-away function. In addition, the tip 1106 of the access needle 1107 can be contrasting tip 1106 as radiopaque and echogenic to facilitate fluoroscopic, cone-beam CT, CT, and ultrasound guided procedures.

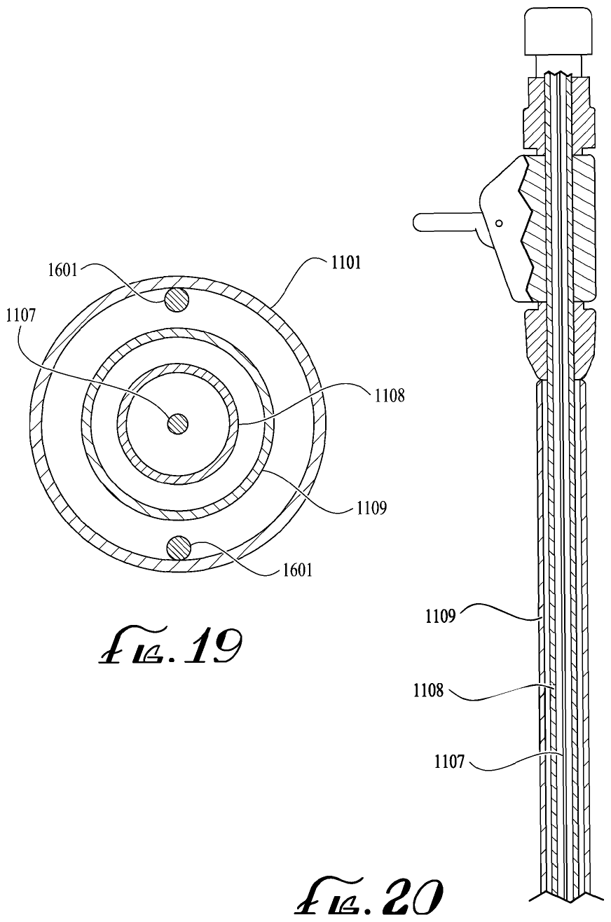

FIG. 13 shows a secondary configuration of the balloon-tipped, peel-away sheath, with insufflation ports located along the shaft of the peel-away sheath rather than along the hubs. Shown in FIG. 13 are sheath 1101, spacer 1102, two ports 1103, hemostatic valve 1104 here shown with Luer lock to flush sheath 1101, and passthrough 1105 as coaxial inner diameter to accommodate access needles, biopsy needles, ablation needles, drainage catheters or other intervention tools. Sheath 1101 can be removable and suitably configured for removal upon acquiring a target. Spacer 1102 can provide blunt dissection prior to unsheathing needle 1107.

FIG. 14 shows a non-insufflated, sheathed configuration of a possible embodiment of the present invention. Shown in FIG. 14 are sheath 1101 and spacers 1102. Spacers 1102 are shown as not insufflated (FIG. 14). Spacers 1102 can be non-insufflated balloons and thus have not yet been expanded to activate spacing (FIG. 14).

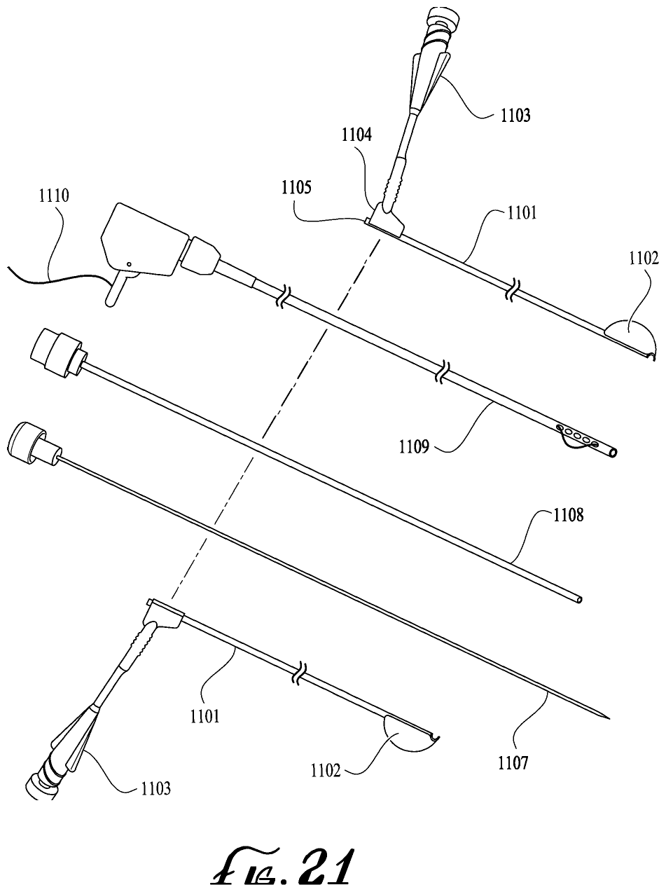

FIG. 15 shows a sheathed configuration with insufflated balloons of an insufflated configuration of a possible embodiment of the present invention. Spacers 1102 can be insufflated (FIG. 15) and needle 1107 can be retracted within sheath 1101.

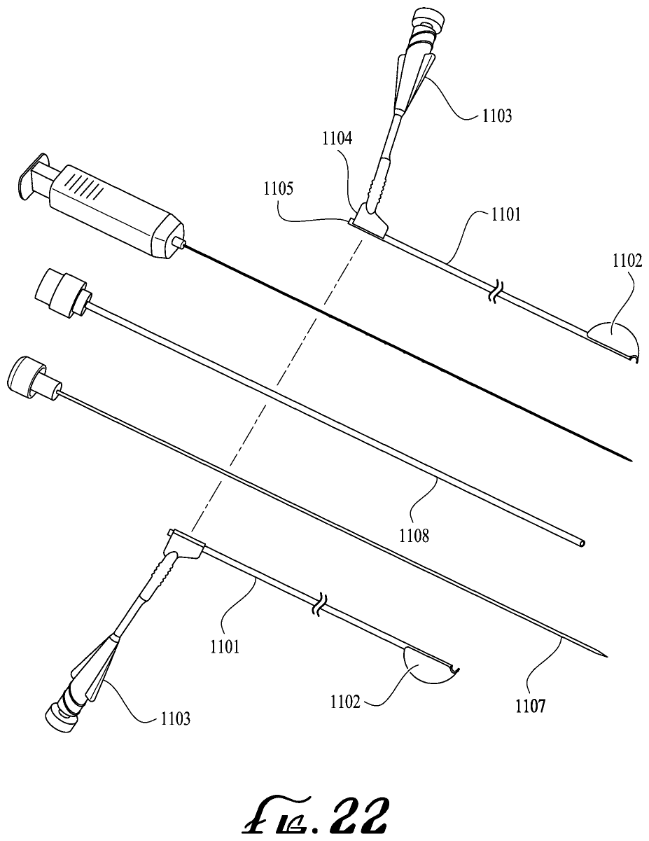

FIG. 16 shows a cut-away view of the balloon-tipped, peel away sheath with coaxial internal drainage catheter, metallic stiffener trocar, sharp inner needle, and insufflation tubing. Tubing 1601 is shown in FIG. 16 with sheath 1101, a pair of spacers 1102, a pair of corresponding ports 1103, tube 1601 providing insufflation from insufflation port 1103 to balloon spacer 1102 and second tube 1601 providing insufflation from to second insufflation port 1103 to second balloon spacer 1102.

Tubing 1601 can be composed of a single tube capable of facilitating insufflation including dilute contrast. Tubing 1601 can provide inner insufflation tubing. In some embodiments, tubing 1601 can provide dual tubing 1601. In many preferred embodiments, tubing 1601 can extend from insufflator port 1103 to inflatable sheath balloon spacer 1102.

FIG. 17 shows a magnified cutaway view of trocar with inner needle having pointed tip therein.

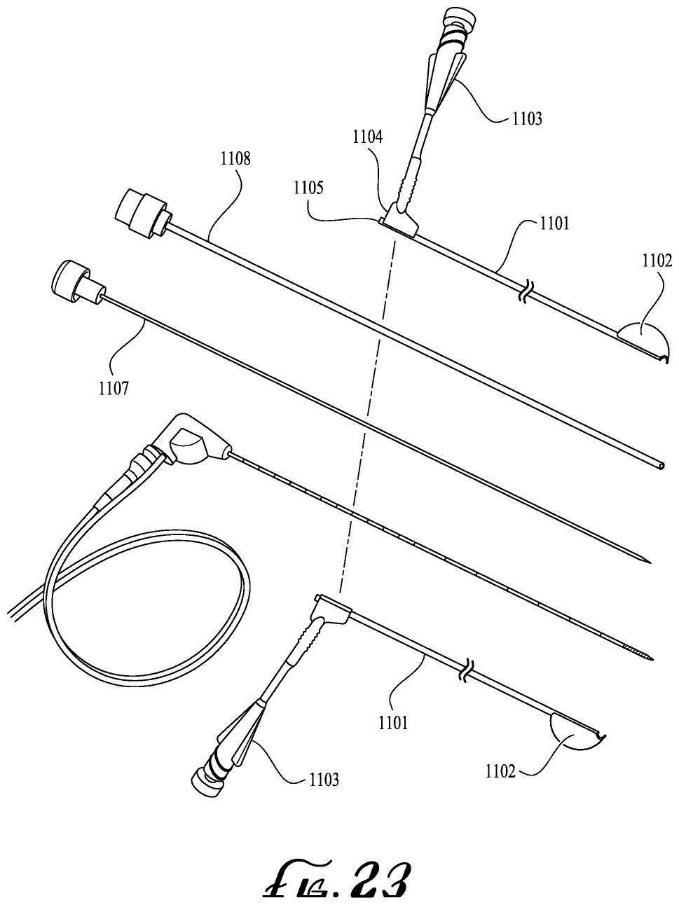

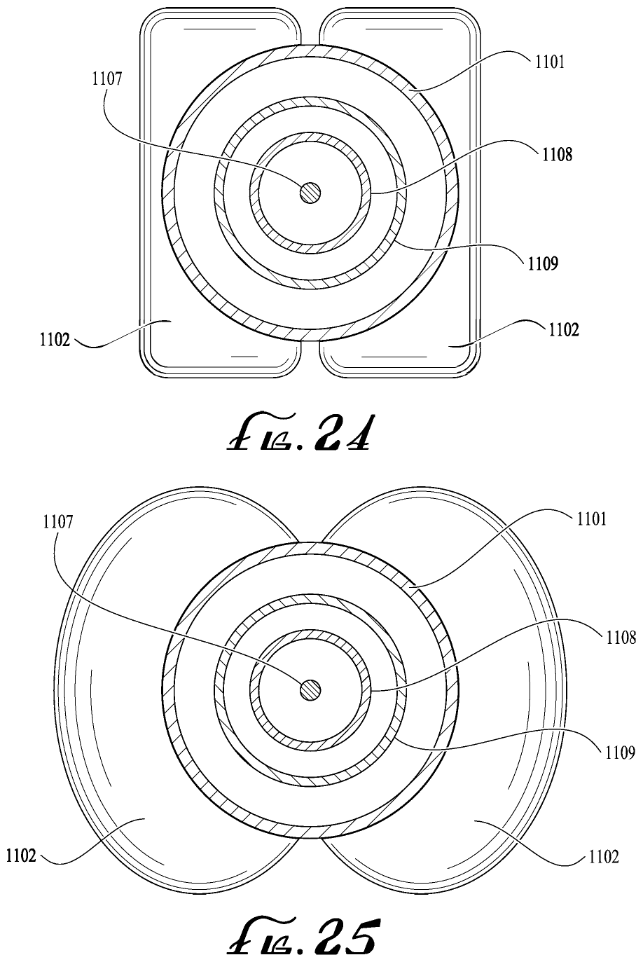

FIG. 18 shows a multilevel cut-away view of the shaft of the device displaying the coaxial nature of the device. A cross-section 19 refers to FIG. 19.

FIG. 19 shows a cross sectional view of the device shaft depicting the coaxial components. Shown in FIG. 19 are sheath 1101, needle with needle 1107, trocar 1108, and two tubes 1601 within sheath 1101 and outside catheter 1109. Tubes 1601 as shown can be longitudinally in opposition along sheath 1101 and parallel to sheath 1101. Tubes 1601 can be inside the diameter of sheath 1101 and outside the diameter of catheter 1109.

FIG. 20 shows a cut away view along the shaft of the pigtail drainage catheter depicting the coaxial nature of the device.

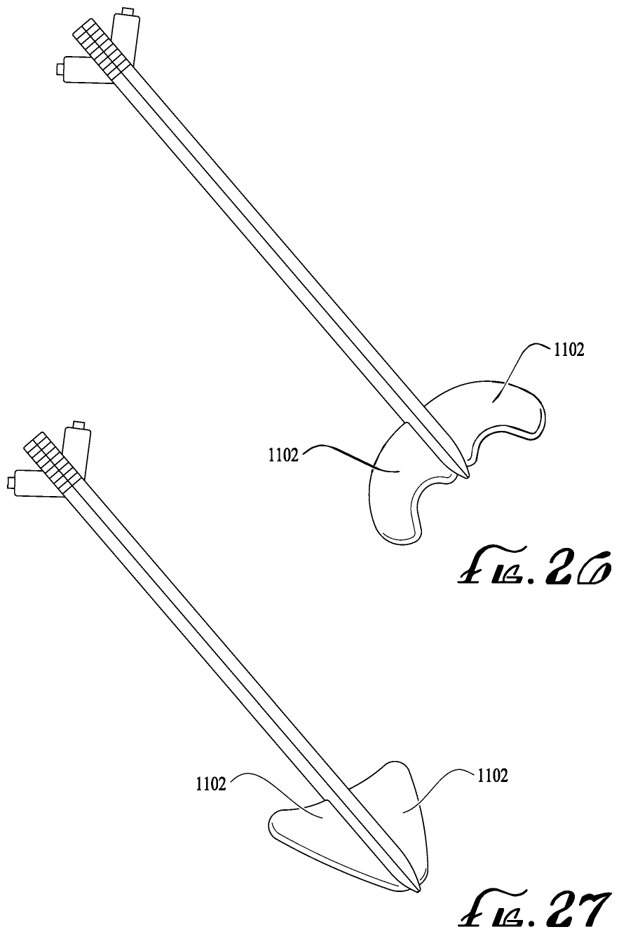

FIG. 21 shows an exploded view of an embodiment of the present invention. Shown in FIG. 21 are sheath 1101, two spacers 1102, insufflation ports 1103, hemostatic valve 1104, catheter 1109, trocar 1108, needle 1107. In a possible embodiment, sheath 1101 can be a peel-away, balloon tipped sheath with the inner drainage pigtail catheter 1109 positioned alongside the coaxial metallic inner stiffener trocar 1108, and sharp inner metallic needle 1107 along with retention suture 1110 as shown in FIG. 21.

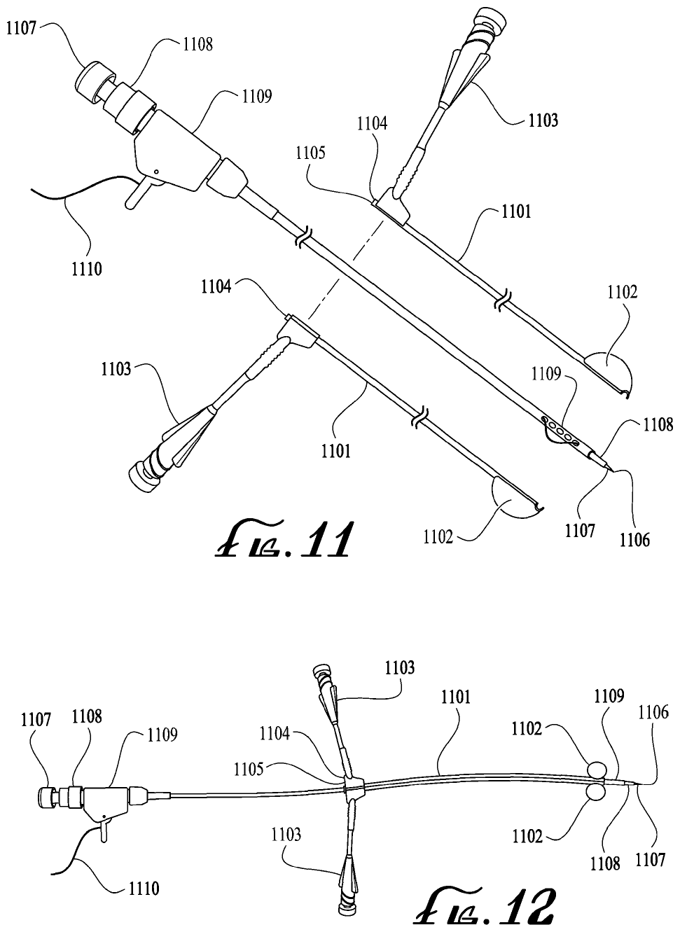

FIG. 22 shows compatibility of the peel-away balloon tipped sheath with a percutaneous biopsy needle device, FIG. 7 referenced art.

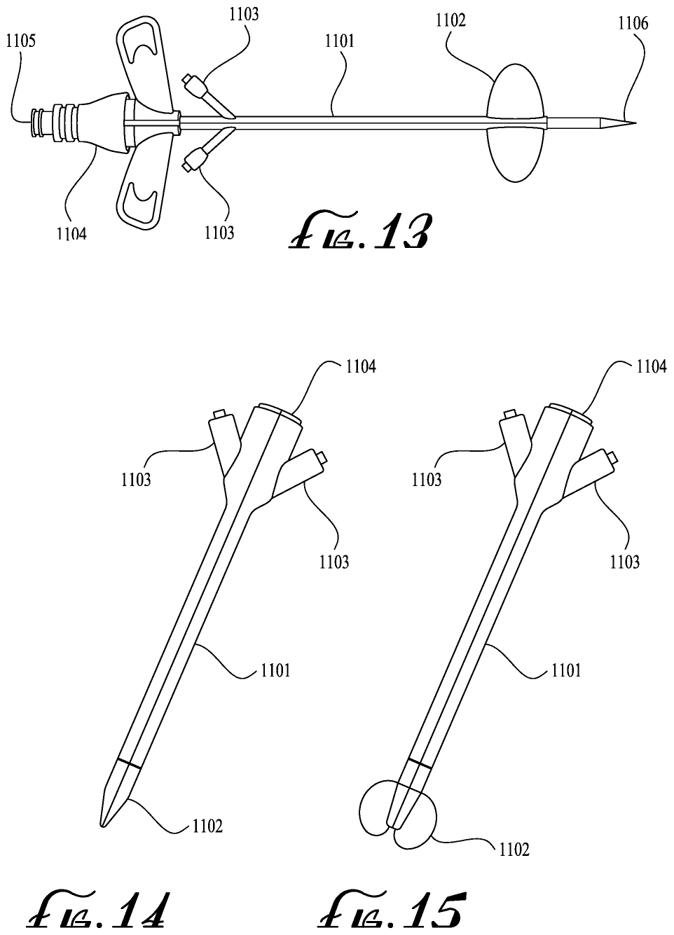

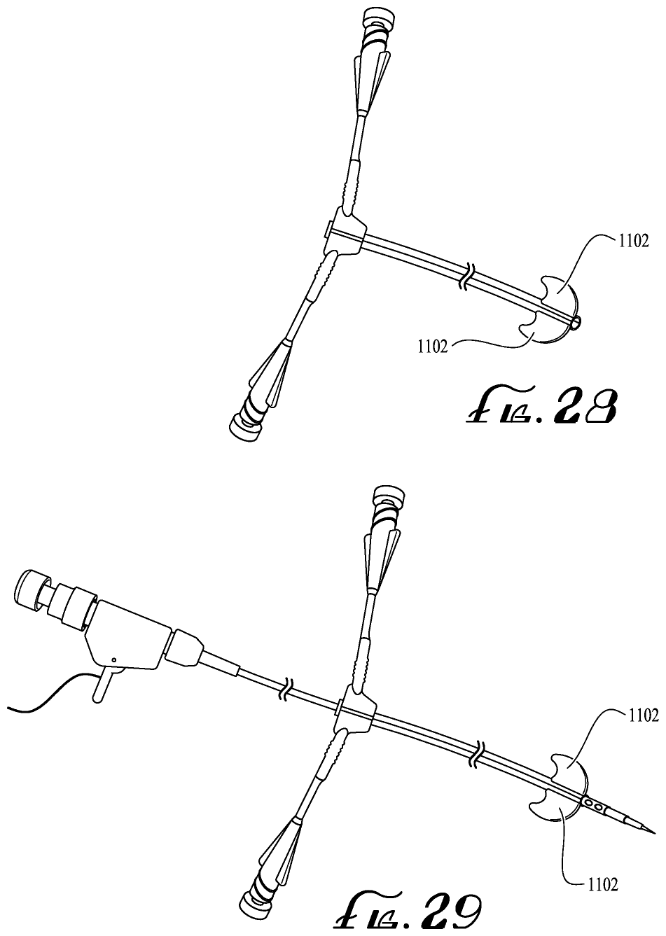

FIG. 23 shows compatibility of the peel-away balloon tipped sheath with an ablation needle probe, FIG. 8 referenced art.

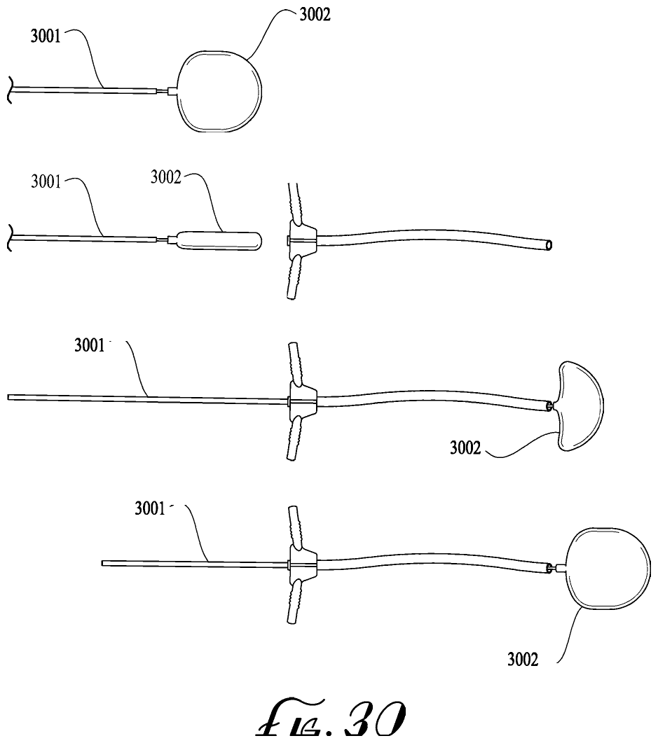

FIG. 24 shows a cross sectional view of the peel away sheath 1101 with insufflated balloons 1102 used coaxially with a pigtail catheter 1109, metallic inner stiffener trocar 1108, and sharp metallic needle 1107.

FIG. 25 shows a cross sectional view of the peel away sheath 1101 with insufflated balloons 1102 used coaxially with a pigtail catheter 1109, metallic inner stiffener trocar 1108, and sharp metallic needle 1107.

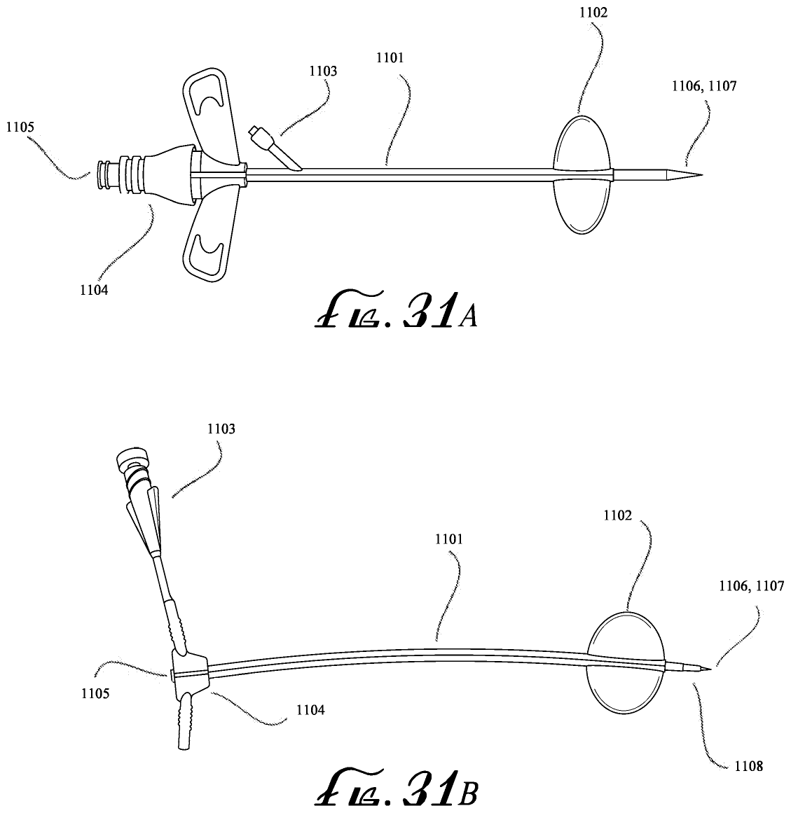

FIG. 26 a view of a possible embodiment of the present invention providing various shapes of spacer 1102.

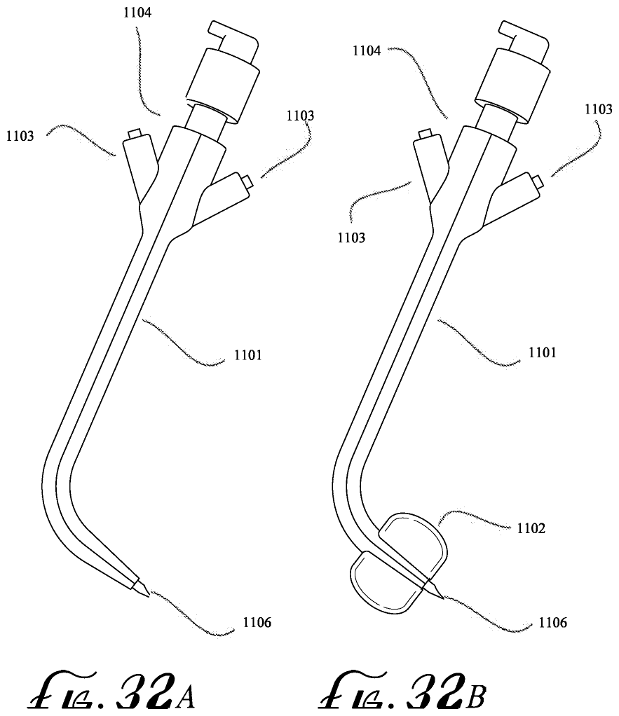

FIG. 27 is a view of a possible embodiment of the present invention providing various shapes of spacer 1102.

FIG. 28 is a view of a possible embodiment of the present invention providing various shapes of spacer 1102.

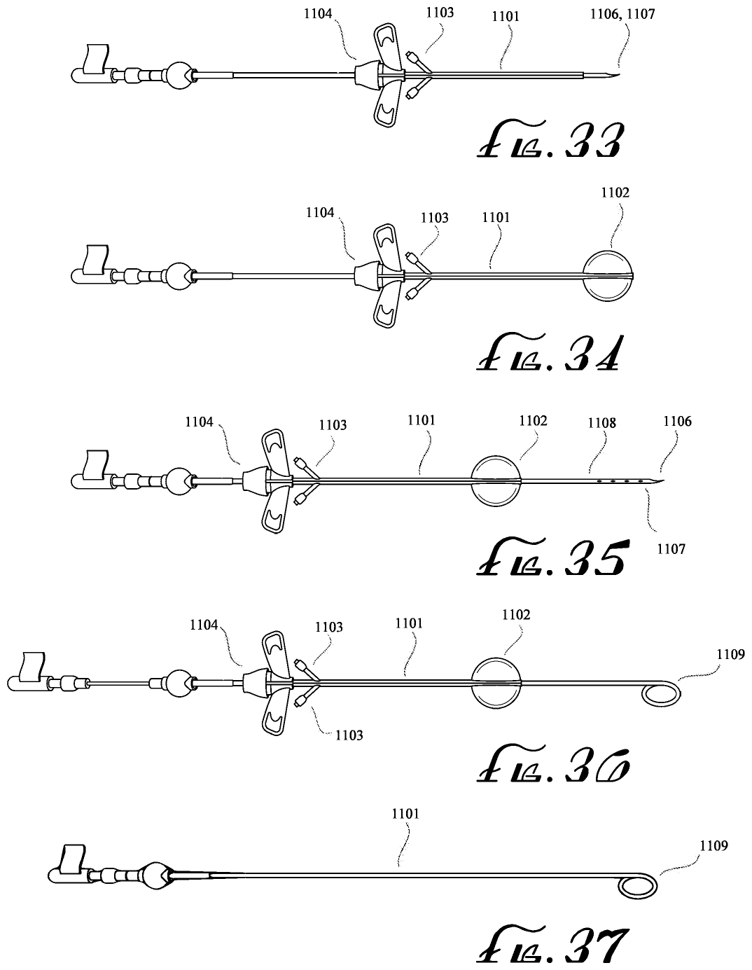

FIG. 29 is a view of a possible embodiment of the present invention with different shaped balloon tip with the coaxial drain, hollow metallic stiffener trocar 1108, and sharp metallic needle as needle 1107.

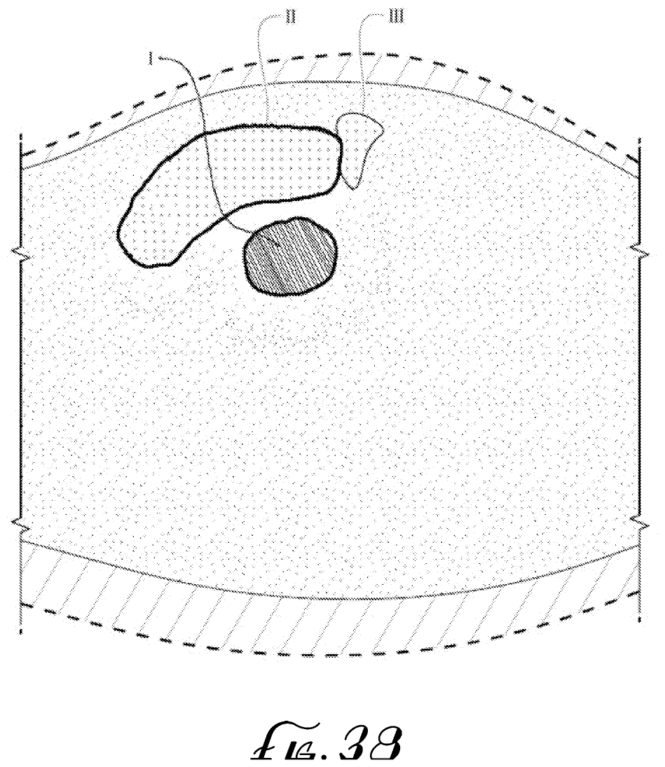

FIG. 30 shows four examples of stages of alternative possible embodiments of the present invention configured for blunt dissection using a shaft 3001 and blunt dissection tip 3002. Blunt dissection tip 3002 can be placed through a non-balloon tipped shaft 3001, including but not limited to peel-away sheaths 1101.

Shown in FIG. 30 are shaft 3001 and blunt dissection tip 3002. Insufflation port 1103 can be absent in embodiments where spacer 1102 is configured with memory properties to form an expanded shape (FIG. 30).

Blunt dissection tip 3002, which can be placed through a non-balloon tipped sheath, including but not limited to peel-away sheaths. Sheath 3001 can be a stiff metallic shaft and a memory-shaped tip composed of metal, mesh, or plastic. This memory-shaped tip can be advanced through a sheath into the abdominopelvic cavity to be used for blunt dissection, with subsequent removal and exchange of the blunt dissection device once the tip of the sheath has reached the target.

Shaft 3001 can be stiff. Shaft 3001 can be flexible and shapeable. Shaft 3001 can be torque-able and steerable. Shaft 3001 can be used for coaxial advancement through a sheath. Shaft 3001 can incorporate an insufflation port for balloon insufflation. Shaft 3001 can be embody sheath 1101.

Blunt dissection tip 3002 can be composed of metallic or alloy compounds such as nitinol. Blunt dissection tip 3002 can be composed of woven mesh. Blunt dissection tip 3002 can be composed of plastics including but not limited to polyurethane. Blunt dissection tip 3002 can be deformed to be placed through a sheath. Blunt dissection tip 3002 can display memory properties and return to its shape despite deformation. In an embodiment, blunt dissection tip 3002 can have a balloon. Blunt dissection tip 3002 with memory reformation properties can be composed of metal, mesh, or plastic. In such embodiments, memory-shaped tip 3002 can be advanced through a sheath into the abdominopelvic cavity to be used for blunt dissection, with subsequent removal and exchange of blunt dissection tip 3002 once the end of sheath 3001 has reached the target. Blunt dissection tip 3002 can thus provide an alternative embodiment of spacer 1102.

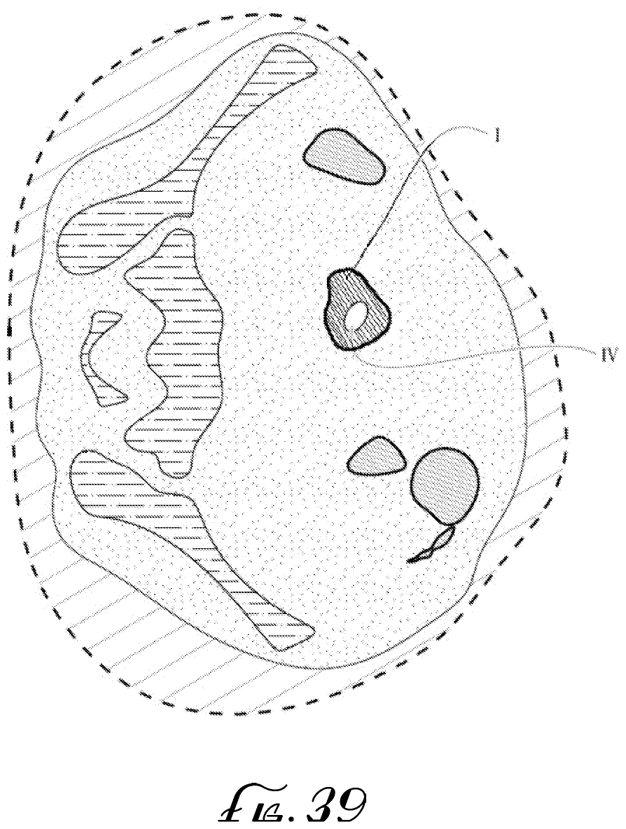

FIG. 31A shows a possible embodiment of the present invention with steerability by which the curve of access sheath 1101 can be manipulated to safely access any anatomic target. As shown in FIG. 31A there can be an absence of balloon-dissecting tip spacer 1102.

FIG. 31B shows a possible embodiment of the present invention with steerability with presence of balloon-dissecting tip spacer 1102 by which the curve of access sheath 1101 can be manipulated to safely access any anatomic target.

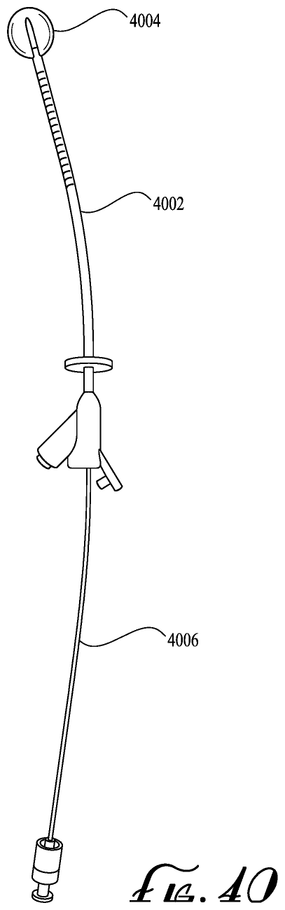

FIGS. 32A and 32B show two iterations of some possible embodiments of the present invention with a single insufflation port 1103 with or without a peel away sheath 1101.

FIG. 32A shows a possible embodiment of the present invention with insufflation port and the absence of a peel away sheath.

FIG. 32B shows a possible embodiment of the present invention with insufflation port with peel away sheath.

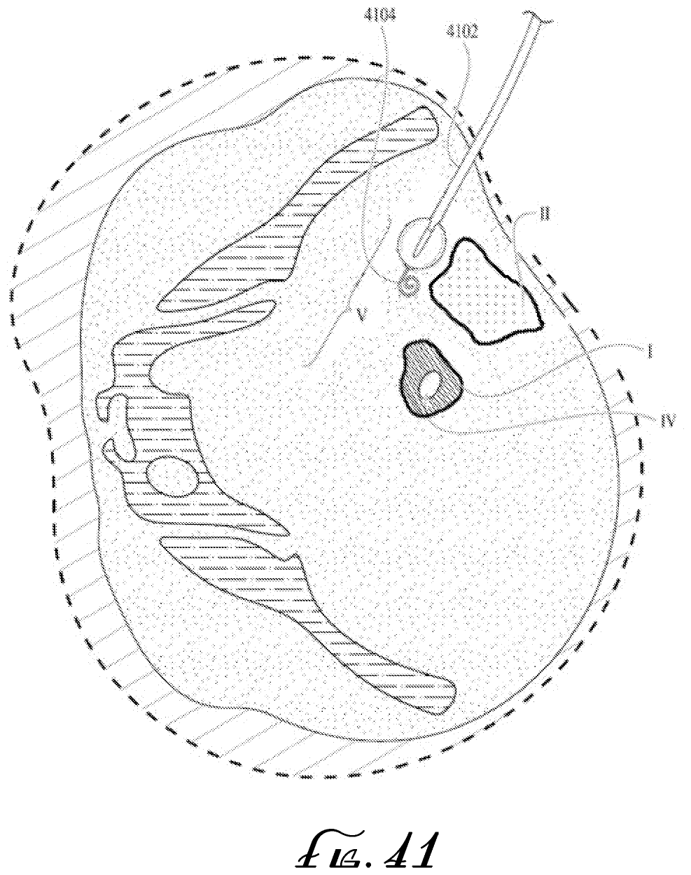

FIG. 33 shows a view of a possible embodiment of the present invention in an unsheathed configuration. Shown in FIG. 33 is needle 1107 to facilitate access, with coaxial hollow metallic stiffener trocar 1108, pigtail drainage catheter 1109, peel-away blunt dissection sheath 1101, with no balloon insufflation spacer 1102. By way of non-limiting illustration, access can be into the abdominopelvic cavity.

FIG. 34 shows a notably withdrawn configuration of a possible embodiment of the present invention without needle 1107 while simultaneously having needle 1107 ready to be extended once the target is reached; and simultaneously spacer 1102 is expanded, in this non-limited illustration, spacer 1102 can be insufflated. A benefit of withdrawn or retracted needle 1107 without pointed tip exposed in FIG. 34 configuration can be to avoid damage to tissue. Shown in FIG. 34 are sheath 1101, spacer 1102 absence or withdrawal of needle 1107, trocar 1108, and catheter 1109. In a preferred embodiment, an optimal blunt dissection configuration can provide needle 1107 with sharp needle being in a withdrawn position; needle 1107 can be retracted into trocar 1108 (FIG. 34). Trocar 1108 can be, in a possible embodiment, formed as a hollow metallic stiffener (FIG. 34) with peel-away, balloon-tipped sheath 1101 (FIG. 34). The balloon tip spacer 1102 (FIG. 34) can be insufflated to facilitate atraumatic blunt dissection.

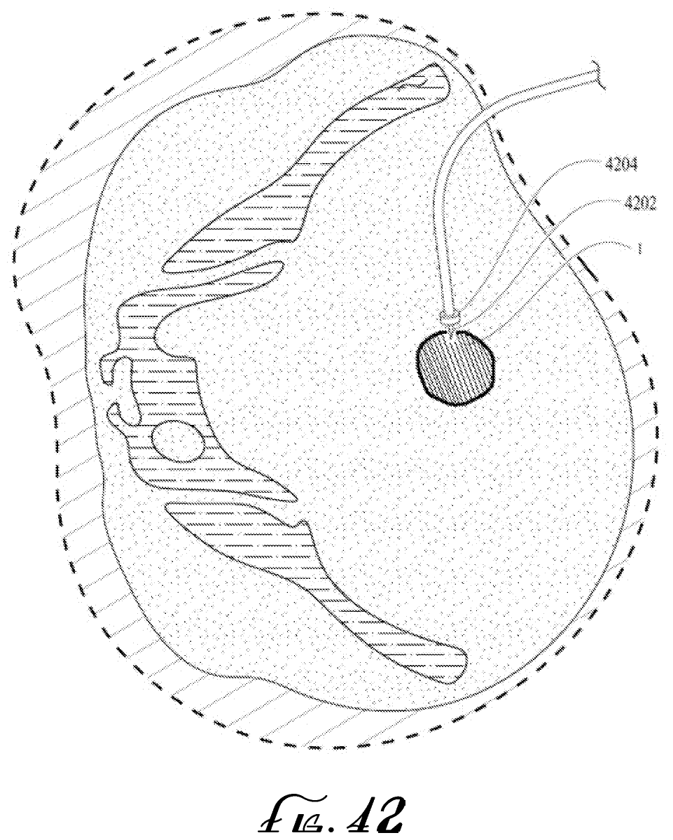

FIG. 35 shows a configuration of a possible embodiment of the present invention. After overlying tissue has been blunt dissected away from the anatomic target, this configuration shows coaxial advancement of the sharp needle tip, metallic stiffener trocar, and pigtail drainage catheter.

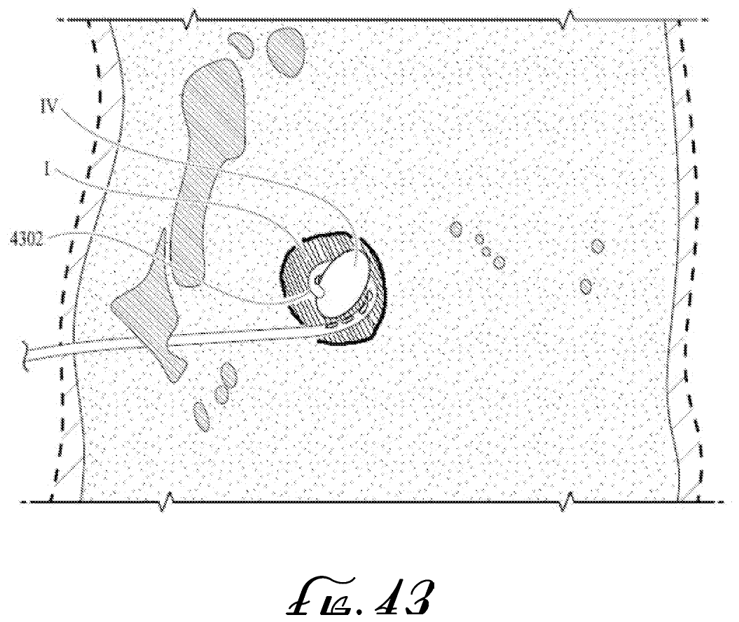

FIG. 36 shows a configuration of a possible embodiment of the present invention. Shown in FIG. 36 are needle 1107, trocar 1108, and catheter 1109. Trocar 1108 can be embodied as a rigid metallic stiffener (FIG. 36). Needle 1107 (FIG. 36) can be a sharp needle tip, and can be slightly withdrawn as shown. Catheter 1109 (FIG. 36) can be a pigtail, multi-side hole, drainage catheter, therein shaped within a target fluid collection.

FIG. 37 shows an alternative configuration of a possible embodiment of the present invention. FIG. 37 shows the needle 1107, hollow stiffening trocar 1108, and peel-away blunt dissection sheath 1101 have been removed, leaving only the drainage catheter 1109 within the target fluid collection.

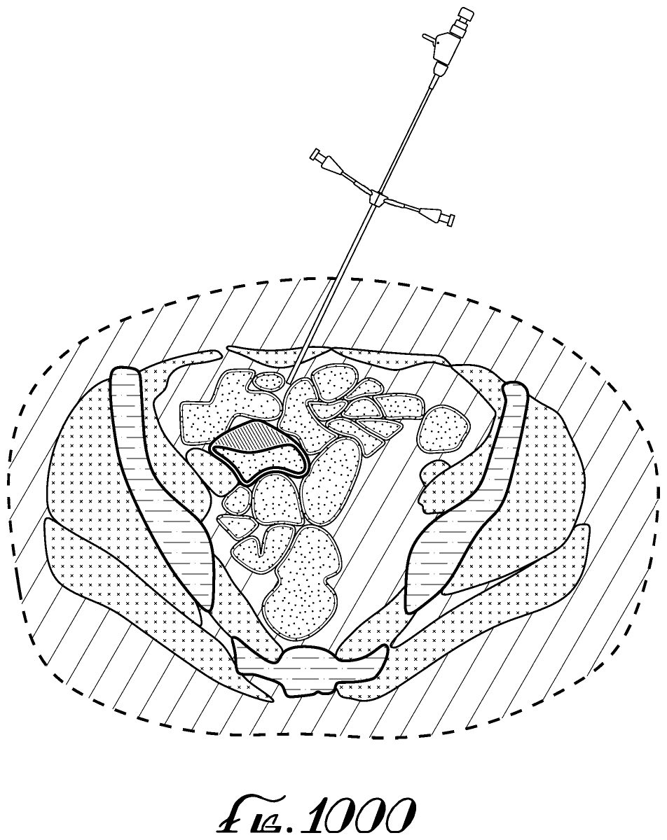

Regarding FIGS. 38-43: In a successful case study, the present invention was successfully used to help treat a patient. In this non-limiting example, patient was a 12-year-old male with no prior medical issues. Patient was sent to emergency department after complaints of pain in lower right side of his abdomen, coupled with fever, nausea and vomiting for past six days. Initial examination revealed vital signs to be normal, however there was tenderness in lower right side of abdomen leukocytosis. Abdominal ultrasound was performed but was inconclusive for appendicitis.

FIG. 38 is a diagram of an image-guided view of pre-procedure conditions with Abscess I is shown in FIG. 38 with cecum II and small bowel III. An MRI scan of the patient revealed a complex collection of fluid in lower right side of abdomen near cecum (FIG. 38). Patient was given broad spectrum antibiotics and was additionally evaluated for percutaneous drain placement, followed by a delayed interval laparoscopic appendectomy. Abdominal MRI scan showed 5.5 cm abscess in pelvic area on right side. There was no direct drainage path from front or back due to intervening cecum and multiple loops of small bowel that bordered abscess. A percutaneous drain placement was performed to try and create a safe pathway, combining hydropneumodissection and blunt dissection. Patient was positioned on his left side to allow cecum and small bowel to move away from abscess. A pre-procedure CT scan showed abscess in lower right side with minimal change in bowel's position. An appendicolith was clearly visible within abscess and served as target for drain placement (FIG. 38). Initially, 21 G introducer needle (MAK-NV; Merit Medical, South Jordan Utah) was used to access space around the abscess. Combination of 20 ml of air and 20 mL of sterile saline was injected to create pathway for wire. Resulting space would allow 0.018″ microwire to enter; however, right colon remained close to abscess. This microwire was slowly advanced into right paracolic gutter, followed by 0.035″ j-tipped wire. Percutaneous tract was serially dilated up to 12-Fr.

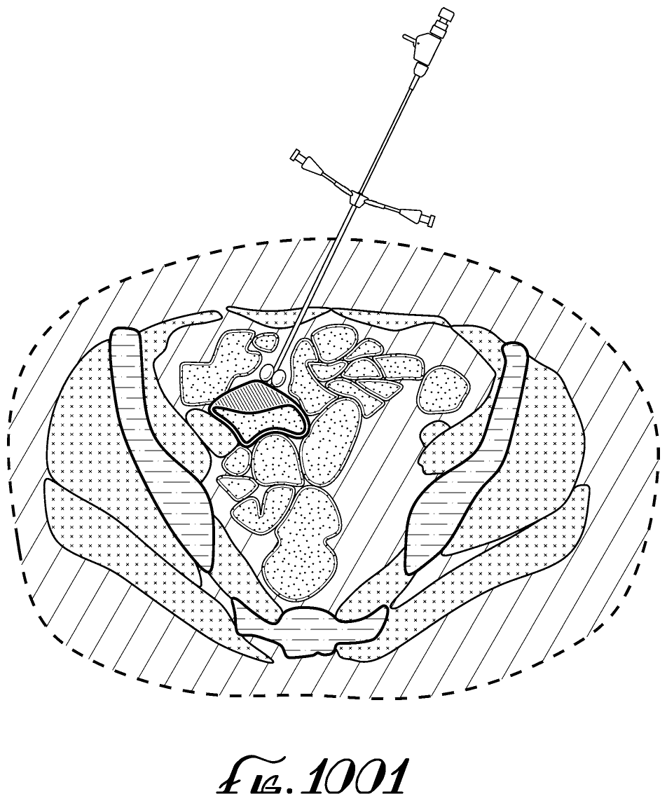

FIG. 39 shows a diagram of an image-guided view of pre-procedure conditions. Abscess I is shown in FIG. 39 with appendicolith IV. An appendicolith was clearly visible within abscess and served as target for drain placement (FIG. 39).

FIG. 40 shows an embodiment of the present invention. Stoma measuring device 4002 is shown in FIG. 40 with inflated balloon tip 4004 and inner metallic stiffener 4006. An additional 10 mL of air and 10 mL of saline was administered into right paracolic gutter which further displaced bowel anteromedially, allowing for blunt dissection and advancement of stoma measuring device (FIG. 40).

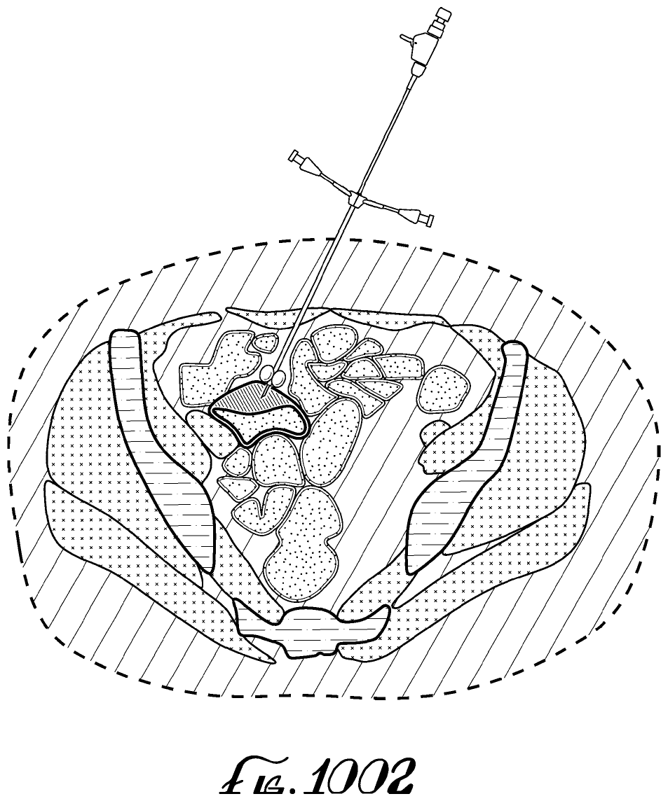

FIG. 41 shows an embodiment of the present invention. Abscess I is shown in FIG. 41 with large bowel or cecum II, appendicolith IV, iliopsoas muscle V, blunt dissection system 4102 and wire 4104.

Blunt dissection system 4102 can comprise a stoma measuring device (MIC-KEY; Avanos, Alpharetta, Georgia) and an inner metal stiffening cannula from a multipurpose drainage catheter set (Cook Medical, Bloomington, Indiana). See FIG. 39. This was advanced over wire into the right paracolic gutter along the anterior margin of the right iliopsoas muscle. Balloon of stoma measuring device was inflated with dilute contrast, to allow for adequate visualization under CT.

Wire 4104 can be a floppy tipped wire 4104 capable of forming a curl shape. Wire 4104, in a preferred embodiment, can be an Amplatz wire. This process was repeated under CT guidance, until stoma measuring device system was positioned adjacent to abscess without intervening bowel. Once safe trajectory was established, 10.2-F, 25-cm multipurpose drain and inner metal stiffening cannula was advanced over wire, with tip directed toward abscess capsule. Inner wire was removed and sharp-tipped stylet was advanced through metal stiffener. Drainage catheter was then placed into abscess with trocar technique (FIG. 41).

FIG. 42 shows an embodiment of the present invention. Sharp tipped stylet 4202 is shown in FIG. 42 with multipurpose drain 4204 and abscess I. Subsequent CT scan was performed demonstrating pig-tail drain terminating within fluid collection (FIG. 42). There were no immediate post procedure adverse events.

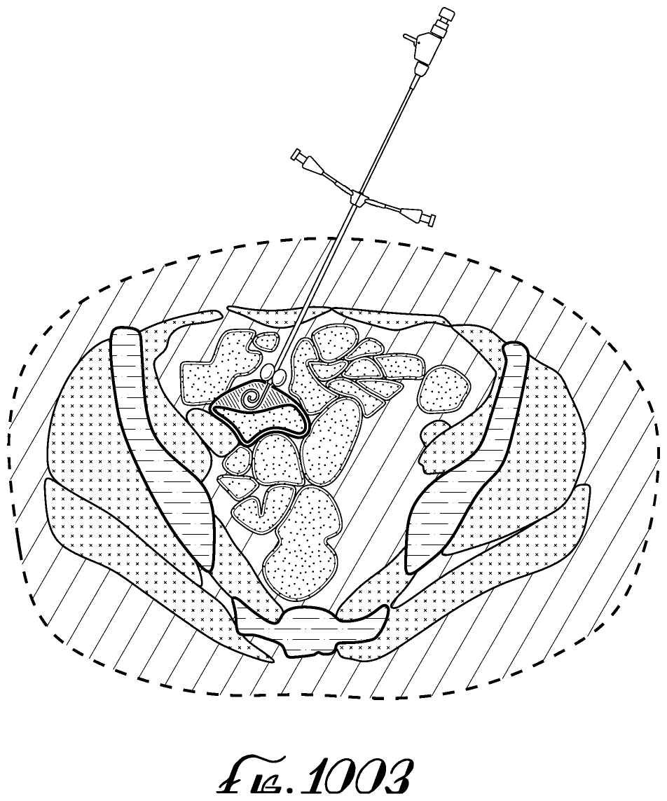

FIG. 43 shows an embodiment of the present invention with multipurpose drain catheter. Visible portions of multipurpose pigtail drain catheter 4302 are shown in FIG. 43 overlapping with abscess I and surrounding appendicolith IV. Patient developed additional abscesses in anterior abdomen and pelvis later in hospitalization, for which additional drains were placed from an anterior and trans gluteal approach. Patient remained afebrile with resolution of his leukocytosis. Patient was later discharged home in stable condition. A benefit of the present invention can be to provide multiple adjunctive techniques, in particular, to make presumed inaccessible fluid collection accessible for safe drainage, such as: hydrodissection, CT gantry angulation, blunt-tip dissection or curved trocar technique (FIGS. 38-43). The present invention can utilize balloon-mediated blunt dissection to add to existing abscess drainage armamentarium. This system can also be achieved through devices readily available in most interventional radiology departments, but has not yet been adopted as such.

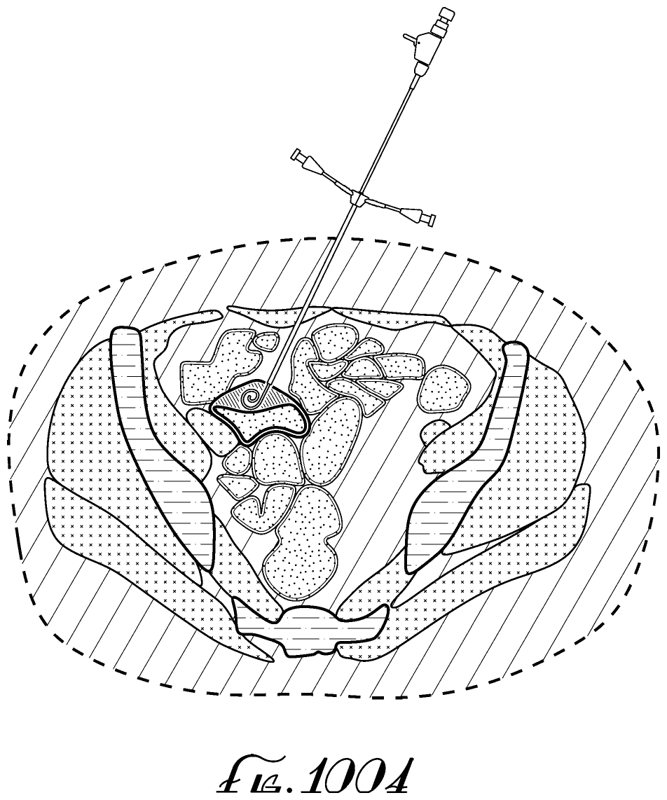

FIG. 1000 demonstrates an example utilization of the device for an intra-abdominal abscess surrounded by multiple loops of bowel, and the first procedural step using the peel-away balloon tipped catheter in a possible embodiment of the present invention.

FIG. 1001 demonstrates inflation of the balloon-dissecting tip, enabling blunt dissection and safe advancement of the device towards the abscess in a possible embodiment of the present invention.

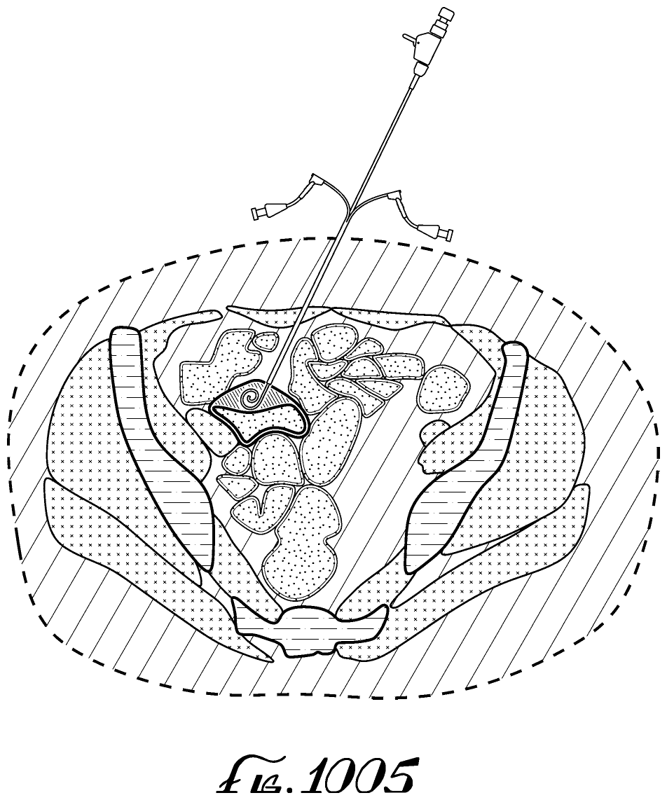

FIG. 1002 shows advancement of a sharp coaxial needle once blunt dissection has moved nontarget tissue out of the way and the tip of the sheath can be immediately adjacent to the target tissue in a possible embodiment of the present invention.

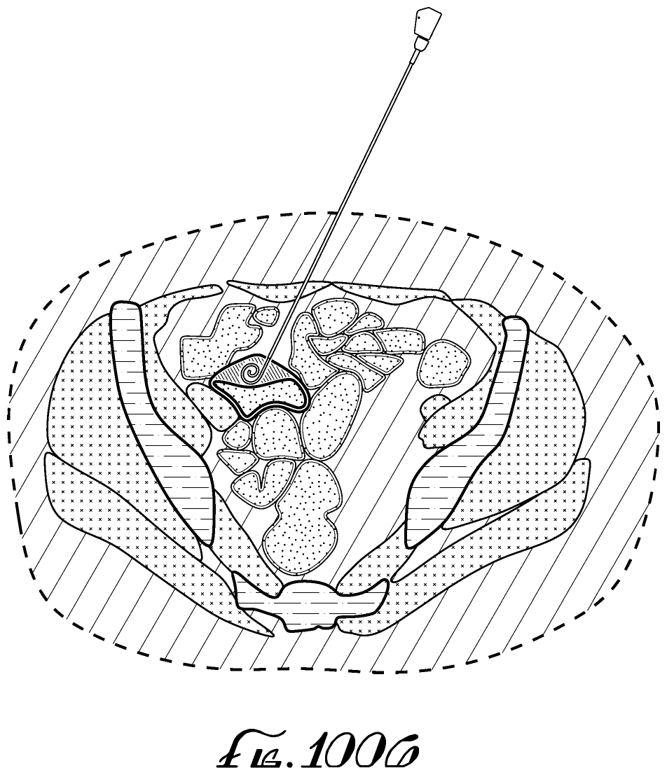

FIG. 1003 shows advancement of the pigtail catheter into the target tissue or target abscess in a possible embodiment of the present invention.

FIG. 1004 shows deflation of spacers 1102 can facilitate the peel-away removal of the sheath in a possible embodiment of the present invention.

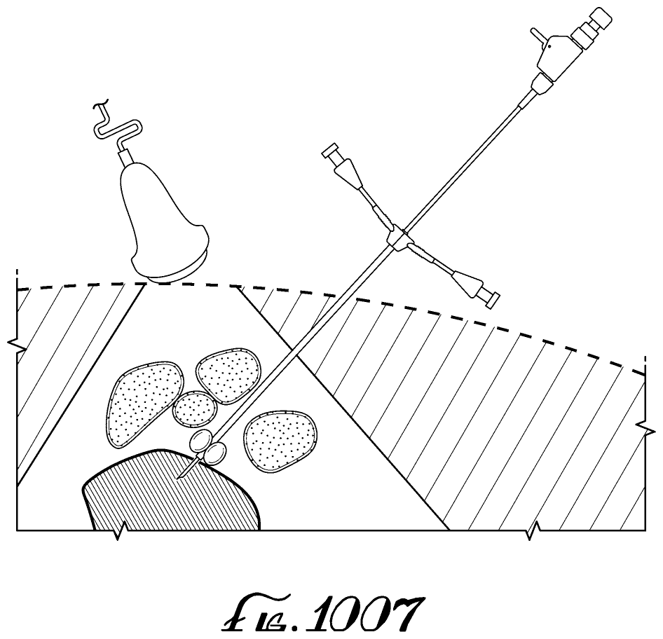

FIG. 1005 shows a further step of peel away removal of sheath 1101 in a possible embodiment of the present invention.

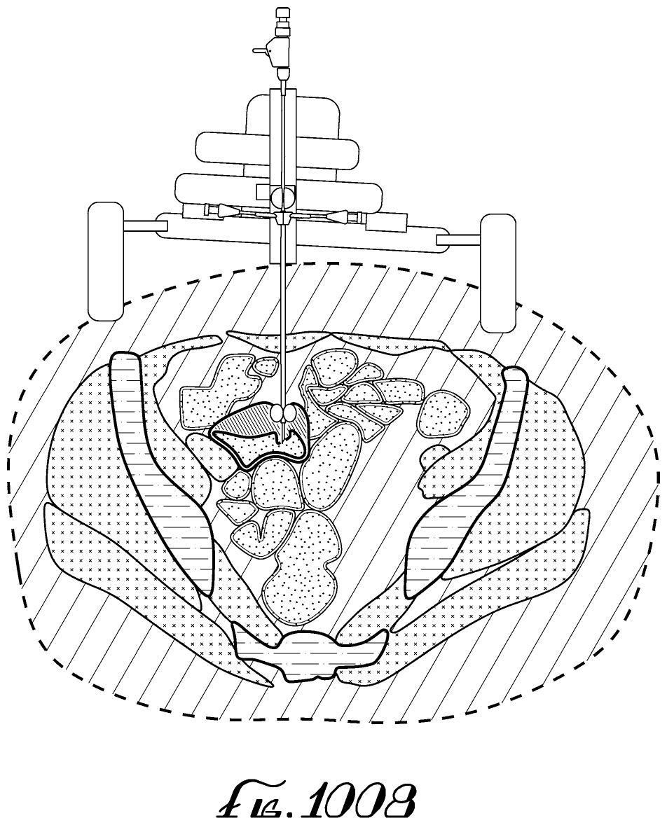

FIG. 1006 shows a subsequent step with drainage catheter 1109 present a possible embodiment of the present invention.

FIG. 1007 shows ultrasound guided access of target tissue using peel-away, balloon tip sheath 1101 in a possible embodiment of the present invention.

FIG. 1008 demonstrates compatibility of the blunt dissection sheath with robotic or artificial-intelligence devices (e.g., FIG. 9) in a non-limiting anatomical example demonstrating a possible embodiment of the present invention.

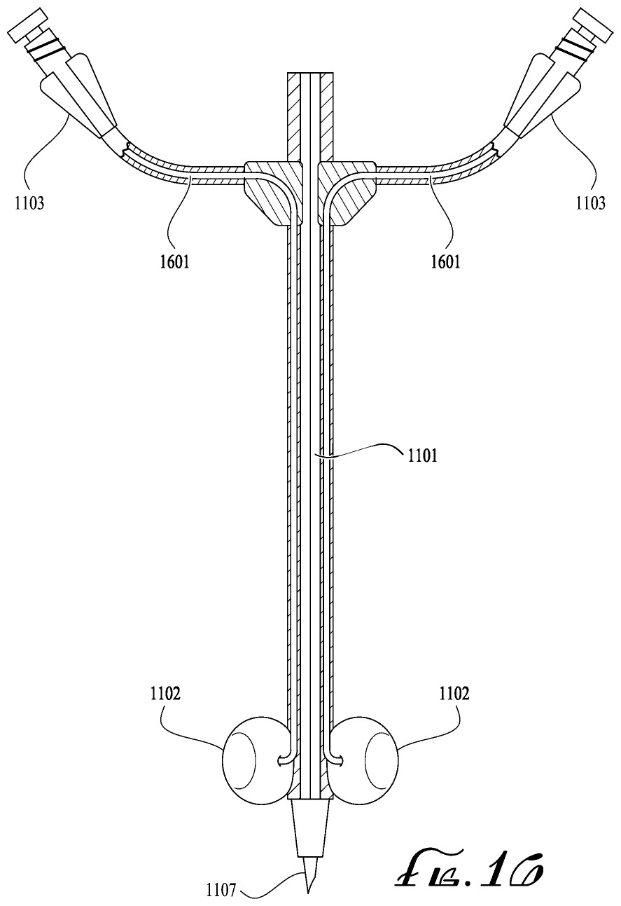

FIG. 1009 demonstrates compatibility of the blunt dissection sheath with an additional robotic or artificial-intelligence device (e.g., FIG. 10) in a non-limiting anatomical example demonstrating a possible embodiment of the present invention.

A method for commencing an interventional radiology procedure can provide inserting needle, balloon spacer, and sheath percutaneously without initially extending needle; expanding balloon spacer while simultaneously maintaining needle in sheathed position within sheath; and subsequently unsheathing needle through sheath percutaneously. Said method can further comprise removably peeling away a balloon tip sheath from sheath; extending a drainage catheter along sheath to expose drainage catheter through sheath; extending a needle coaxially through access sheath to expose drainage catheter through sheath; extending a drainage catheter through access sheath to expose drainage catheter through sheath; extending an ablative dissection needle through access sheath; performing a biopsy by obtaining tissue from a target site via dissection; performing drainage after placing drainage catheter at target area through sheath; and flushing sheath using a Luer lock; and performing a tumor ablation at target site.

### CONCLUSION

In summary, the present invention provides a percutaneous pathway system and method. The system can have a peel-away coaxial sheath and percutaneous needle access device, with separately inflatable balloons on each half of the peel-away sheath, suitable for obtaining percutaneous access into the abdomen and pelvis with subsequent blunt tissue dissection and/or tissue manipulation.

The foregoing description of the preferred embodiments of the invention has been presented for the purposes of illustration and description. It is not intended to be exhaustive or to limit the invention to the precise form disclosed. Many modifications and variations are possible in light of the above teaching, it is intended that the scope of the invention are not, and need not be, limited by this detailed description, but by the claims and the equivalents to the claims which relate to the present invention. Use of punctuation and any articles “a” or “the” in reference to matter claimed shall be construed broadly to uphold the appended claims and equivalents thereto. This specification shall be construed broadly to uphold the claims and equivalents thereto, as set forth by the claims appended hereto. Any claims appended hereto are deemed part of this disclose and are incorporated herein by reference, and will be understood as self-sufficient to support said claims.

What is claimed is:

1. A percutaneous system, comprising:
a sheath (1101).

2. The system of claim 1, further comprising:
a spacer (1102) is placed upon the sheath (1101).

3. The system of claim 2, further comprising:
a needle (1107) is parallel within the sheath (1101).

4. The system of claim 3, wherein the needle (1107) is sheathed so the needle (1107) is not exposed beyond the spacer (1102).

5. The system of claim 4, further comprising:
a passthrough (1105) wherein the needle (1107) is retractable within the passthrough (1105).

6. The system of claim 5, wherein the needle (1107) is not exposed beyond the spacer (1102) while retracted.

7. The system of claim 6, wherein needle (1107) is extendable beyond the spacer 1102 to suitably acquire a target.

8. The system of claim 2, further comprising:
wherein the spacer (1102) comprises a balloon.

9. The system of claim 8, further comprising:
an at least one insufflation port (1103) on the sheath (1101) to insufflate the balloon of the spacer (1102).

10. The system of claim 9, further comprising:
a passthrough (1105) comprises a coaxial capable inner diameter.

11. The system of claim 3, further comprising:
a hemostatic valve (1104) on the sheath (1101).

12. The system of claim 3, further comprising:
a contrasting tip (1106) formed on the needle (1107), which is suitably configured for image-guided surgical procedures.

13. The system of claim 1, further comprising:
a trocar (1108).

14. The system of claim 3, further comprising:
a catheter (1109) parallel with the sheath (1101).

15. The system of claim 14, wherein the sheath (1101) is coaxial around the passthrough (1105) which serves as an access tube.

16. The system of claim 15, further comprising:
a retention suture (1110) within the catheter (1109) which is capable of curling the catheter (1109).

17. The system of claim 16, wherein the retention suture (1110) facilitates formation of a pigtail loop to drain via a side hole of catheter (1109).

18. A method for commencing an interventional radiology procedure, comprising:
inserting a needle (1107), a balloon spacer (1102), and a sheath (1101) percutaneously without initially extending the needle (1107).

19. The method of claim 18, further comprising:
expanding the balloon spacer (1102) while simultaneously maintaining the needle (1107) in sheathed position within the sheath (1101).

20. The method of claim 19, further comprising:
subsequently unsheathing the needle (1107) through the sheath (1101) percutaneously.
